(19) United States
(12) Patent Application Publication
(10) Pub. No.: US 2006/0073483 A1
(43) Pub. Date: Apr. 6, 2006

(54) Electro-optical nucleic acid-based sensor array and method for detecting analytes

(76) Inventors: Joel E White, Millis, MA (US); John S Kauer, Weston, MA (US)

Correspondence Address: DAVID S. RESNICK, 100 SUMMER STREET, NIXON PEABODY LLP, BOSTON, MA, 02110-2131, US

(21) Appl. No.: 10/535,748

(22) PCT Filed: Nov. 25, 2003
(86) PCT No.: PCT/US03/38186

§ 371 Date: Sep. 27, 2005

(51) Int. Cl. C12Q 1/68; G06F 19/00

(52) U.S. Cl. 435/6; 702/20

(57) ABSTRACT

The present invention is directed to methods of detection, identification and monitoring of vapor phase analytes by using sensor arrays comprising fluorophore labeled nucleic acids, dried onto a substrate which react with vapor phase analytes. Methods of using and preparing such sensor arrays are also provided.

### CROSS-REFERENCE TO RELATED APPLICATIONS

This application claims the benefit of the U.S. Provisional Patent Application Ser. No. 60/428,869, filed Nov. 25, 2002, and a co-pending U.S. patent application Ser. No. 10/303,548 filed Nov. 25, 2002.

### GOVERNMENT SUPPORT

The invention described herein was supported in part with U.S. Government funding under Defense Advance Research Projects Agency Contract No. DAAK60-97-K-9502, Office of Naval Research Grant No. N00014-95-1-1340, and National Institutes of Health Grant DC00228. The U.S. government has certain rights in this invention.

### FIELD OF THE INVENTION

The present invention generally relates to compositions and systems useful in monitoring of chemical hazards, air quality, and medical conditions, and detecting explosives, mines, and hazardous chemicals. The invention provides nucleic acid-based sensors and methods for detecting analytes. More particularly, the invention relates to nucleic acid-based optical sensors, sensor arrays, sensing systems and sensing methods for sensing and detection of unknown analytes in vapor phase by way of real-time feedback and control of sampling conditions including remote controlled systems and methods of making such sensors and sensor arrays.

### BACKGROUND OF THE INVENTION

The serious threat of explosive, chemical and/or biological attacks pose a particular challenge for national security in the current “post September 11th, 2001” era. A method that could detect a wide range of compounds, and that could also be automated and remotely controlled and that could be used in field conditions including airport, seaport, or other screening systems, would be particularly desirable. For example, currently only about 2% of all the containers are screened by any means that come through the seaports to the United States, because there are no suitable reliable, fast, easy and relatively cheap screening methods available. For national security, it is imperative to develop screening methods that could detect, for example, explosives and toxic chemicals that may be transported into the United States. Detection methods for identifying trace amounts of volatile compounds from, for example, explosives or chemical warfare agents, would be one possible way to approach such novel screening methods for national security purposes.

Moreover, there are a number of other current and potential uses for detection and identification of volatile compounds. For example, different chemical analyses have been used to detect the presence or absence of a known target chemical in clinical diagnoses, to identify unknown compounds and mixtures in basic research and drug discovery, and to document the identity and purity of known compounds, e.g., in testing and quality control in drug manufacturing processes. In addition to laboratory analyses, chemical detection is also important outside of the laboratory. Examples include bedside diagnoses, and environmental monitoring for hazardous materials. The “field” applications, including detection of explosives and chemical warfare agents, require small, portable, reliable, easy-to-use, inexpensive devices.

There are number of methods currently available for chemical analysis, each appropriate for a particular application and each having its own strengths and weaknesses. Examples include the various forms of chromatography, including gas chromatography (GC), high performance liquid chromatography (HPLC), and spectroscopy, including mass spectroscopy (MS), ion mobility spectroscopy (IMS), Raman spectroscopy and infrared spectroscopy, as well as other chemical, immunological, and gravimetric methods. Also, combinations of different methods can provide a powerful means of identifying unknown compounds, e.g., GC/MS which is used extensively in analytical chemistry laboratories.

A common feature of these analytical methods is that the chemical sample needs to be prepared prior to analysis. Liquid and solid samples are usually dissolved into an appropriate solvent. For analysis of vapor-phase chemicals, a preconcentration step is often required to increase the quantity of material for analysis.

Preconcentration of vapor-phase chemicals involves passing a large volume of air over an adsorbent Tenax or solid phase microextraction (SPME) trap. The sample is removed from the trap using a small amount of liquid solvent or is thermally desorbed directly into the input of a GC for analysis (Zhang, Z., Yang, M. J., and Pawliszyn, J. (1994) Anal. Chem., 66:844A-853A).

Preconcentration followed by GC or GC/MS has been used to detect and quantify volatile chemicals in a variety of studies with relevance to health care and domestic security, for example, detection of contaminants in water (Lambropoulou, D. A. and Albanis, T. A. (2001) J. Chromatogr., 922:243-255; Cancho, B., Ventura, F., and Galceran, M. T. (2002) J. Chromatogr., 943:1-13) and soils (Cam, D. and Gagni, S. (2001) J. Chromatogr. Sci., 39:481-486), detection of toxic substances in blood (Bouche, M. P., Lambert, W. E., Bocxlaer, J. F. V., Piette, M. H., and Leenheer, A. P. D. (2001) J. Anal. Toxicol., 26:35-42;Musshoff, F., Junker, H., and Madea, B. (2002) J. Chromatogr. Sci., 40:29-34), detection of drugs in postmortem tissue (Mosaddegh, M. H., Richardson, R., Stoddart, R. W., and McClure, J. (2001) Ann. Clin. Biochem., 38:541-547), detection of organic compounds in normal breath (Grote, C. and Pawliszyn, J. (1997) Anal. Chem., 69:587-596) and in the breath of lung cancer patients (Phillips, M., Gleeson, K., Hughes, J. M. B., Greenberg, J., Cataneo, R. N., Baker, L.; and McVay, W. P. (1999) Lancet, 353:1930-1933), characterization of explosive signatures (Jenkins, T. F., Leggett, D. C., Miyares, P. H., Walsh, M. E., Ranney, T. A., Cragin, J. H., and George, V. (2001) Talanta, 54:501-513), and detecting Sarin in air and water (Schneider, J. F., Boparai, A. S., and Reed, L. L. (2001) J. Chromatogr. Sci., 39:420-424). For rapid detection of volatile compounds, it would be advantageous to avoid specific sample preparation steps. This would be especially desirable in applications where detection is performed in field conditions, outside a laboratory.

Volatile chemical analyses using these methods require optimizations for each analysis problem. For example, the GC column, GC detector, trap coatings, and flow rates all need to be optimized for particular volatiles of interest. In addition, preconcentration can take considerable time to collect sufficient material in the trap. The time required depends on the sorbent coating on the trap (different Tenax coatings have different affinities for different chemical compounds) and on the original concentration of sample in the air. Such analytical methods are therefore generally inappropriate for rapid analyses, such as security screening, real-time environmental monitoring, or bedside diagnoses. Therefore, it would be advantageous to develop a detection system that is capable of rapidly analyzing a wide array of different compounds in varying concentrations.

For air sampling, ah alternative to preconcentration consists of systems containing dedicated sensors that are responsive to particular compounds of interest. Common examples include home detectors for carbon monoxide, propane, and natural gas. Although sensors are available that are broadly responsive, e.g., sensors that respond to many volatile organic compounds, these devices do not identify the vapor detected. While a system containing a dedicated selective sensor can respond rapidly to its cognate analyte and may not require preconcentration, the ability to detect and identify multiple volatile compounds would require a separate sensor selective for each compound of interest. Further, such methods preclude detection of future compounds of interest. Therefore, it would be desirable to develop a system that is capable of sensing as well as identifying a wide range of compounds.

For detecting, discriminating, and identifying volatile compounds in the air, one of the most highly developed chemical detection devices is arguably the olfactory system of terrestrial animals. Olfactory abilities include high sensitivity (Passe, D. H. and Walker, J. C. (1985) Neurosci. Biobehav. Rev., 9:431-467), the ability to detect and discriminate many different compounds (e.g., Youngentob, S. L., Markert, L. M., Mozell, M. M., and Homung, D. E. (1990) Physiol. Behav., 47:1053-1059; Slotnick, B. M., Kufera, A., and Silberberg, A. M. (1991) Physiol. Behav., 50:555-561; Lu, X.-C. M., Slotnick, B. M., and Silberberg, A. M. (1993) Physiol. Behav., 53:795-804), and the ability to make fine odorant discriminations (e.g., individual recognition in rodents—Yamazaki, K., Singer, A., and Beauchamp, G. K. (1998-1999) Genetica, 104:235-240). Numerous mechanisms influence these capabilities at points in the process even before odorant molecules interact with receptor proteins. Sniffing behavior, nasal aerodynamics, mucus solvation, and odorant clearing all likely play a role in olfactory abilities (Christensen, T. A. and White, J. (2000). Representation of olfactory information in the brain. In Finger, T. E., Silver, W. L., and Restrepo, D., editors, Neurobiology of Taste and Smell, pages 201-232. John Wiley & Sons, New York). Once odorants reach the olfactory receptor proteins in the nasal mucosa, the system does not devote one receptor protein to each individual odorous ligand. Rather, even single compounds interact with many broadly-responsive receptor proteins, producing widespread spatiotemporal patterns of activity in the olfactory sensory neuron population—in other words, activity in many sensor elements that evolve over time (MacKay-Sim, A., Shaman, P., and Moulton, D. G. (1982) J. Neurophysiol., 48:584-596; Kent, P. F. and Mozell, M. M. (1992) J Neurophysiol, 68:1804-1819). These patterns of activity are then interpreted by parallel processing elements in the olfactory areas of the brain, producing widespread activation in the neuronal populations at each level of the olfactory pathway (for reviews, see Kauer, J. S. (1987). Coding in the olfactory system. In Finger, T. E. and Silver, W. L., editors, Neurobiology of Taste and Smell, pages 205-231. John Wiley & Sons, Inc, New York; Kauer, J. s. (1991). Trends Neurosci, 14:79-85; Christensen, T. A. and White, J. (2000). Representation of olfactory information in the brain. In Finger, T. E., Silver, W. L., and Restrepo, D., editors, Neurobiology of Taste and Smell, pages 201-232. John Wiley & Sons, New York).

The properties of the olfactory system suggest that engineered devices based on olfactory mechanisms may have advantages for detecting and identifying volatile compounds. Such a device, called an “artificial nose” or “electronic nose,” was first described in the early 1980's (Persaud, K. and Dodd, G. (1982). Nature, 299:352-355), and a number of systems have been reported since then (see, e.g., Grate, J. W., Rose-Pehrsson, S. L., Venezky, D. L., Klusty, M., and Wohltjen, H. (1993) Anal. Chem., 65:1868-1881; Freund, M. S. and Lewis, N. S. (1995). Proc. Nat. Acad. Sci. USA, 92:2652-2656; White, J., Kauer, J. S., Dickinson, T. A., and Walt, D. R. (1996) Anal. Chem., 68:2191-2202).

All of these devices incorporate the two main features that define an electronic nose: 1) an array of broadly-responsive sensors and 2) a pattern recognition method for processing sensor output. Like in the olfactory system, odorants interact with multiple sensors, producing a pattern of activation across the array. Commercial and research electronic noses use a variety of technologies for chemical sensing including conducting polymers, surface acoustic wave devices, solid-state devices, and optical interrogation. Pattern recognition methods generally involve statistical methods or computational neural networks (for reviews, see Gardner, J. W. and Bartlett, P. N., editors (1992). Sensors and sensory systems for an electronic nose. Kluwer Academic Publishers, Dordrecht, The Netherlands; Gardner, J. W. and Bartlett, P. N. (1994). Sensors and Actuators B, 18-19:211-220; Gardner, J. W. and Hines, E. L. (1997). Pattern analysis techniques. In Kress-Rogers, E., editor, Handbook of Biosensors and Electronic Noses: Medicine, Food, and the Environment, pages 633-652. CRC Press, BocaRaton, Fla.; Dickinson, T. A., White, J., Kauer, J. S., and Walt, D. R. (1998). Trends Biotechnol., 16:250-258).

Potential and actual uses of commercial electronic noses include food/beverage analysis, environmental monitoring, and medical diagnosis (Ping, W., Yi, T., Haibao, X., and Farong, S. (1997) Biosens. Bioelectron., 12:1031-1036; Dickinson, T. A., White, J., Kauer, J. S., and Walt, D. R. (1998) Trends Biotechnol., 16:250-258; Aathithan, S., Plant, J. C., Chaudry, A. N., and French, G. L. (2001) J. Clin. Microbiol., 39:2590-2593).

Vapor phase chemical detection systems based on arrays of broadly-responsive sensors offer a number of potential advantages over traditional analytical devices. An electronic nose directly samples the air, so no sample preparation is necessary. The time required for detection is limited only by the time required for the chemical sensors to respond and for the pattern recognition calculation, which is fast using modem computer technology. With rapidly-responding sensors, rapid detection of volatiles is therefore possible. In addition, while traditional analytical instruments tend to be large and require considerable power, sensor array devices have the potential for being small and portable. Although handheld IMS devices are available, they are currently tuned to specific, restricted tasks, such as use of the Iontrack Instruments VaporTracer2 for explosives or drugs, and therefore lack the broad-band nature of an electronic nose.

Sensor array devices would also have a number of advantages over systems using mono-specific sensors. First, truly “mono-specific” sensors are difficult (if not impossible) to produce; broadly-responsive sensors can be readily made. Second, even if mono-specificity could be achieved, detection of several compounds would require development of a separate sensor for each compound of interest. Conversely, a relatively small array of broadly-responsive sensors is theoretically capable of discriminating a large number of different compounds (Alkasab, T. K., White, J., and Kauer, J. S. (2002) Chem. Senses, 27:261-275). Third, a device containing sensors specific for a finite number of compounds is incapable of detecting any others outside its defined target set. A device containing broadly-responsive sensors would have the potential for detecting and discriminating compounds of future interest.

It would be advantageous to develop sensors capable of detecting and correctly identifying a large range of analytes, e.g., volatile chemicals. Such sensors would be particularly useful in domestic security applications, such as detecting explosives and chemical warfare agents.

### SUMMARY OF THE INVENTION

We have, surprisingly, discovered that nucleic acids with attached fluorophores and dried onto a substrate react with volatile chemical compounds or analytes in ambient air and can therefore be used as sensors to detect analytes in the air that react thereto. This is distinctly different from other nucleic acid-based sensing materials that work only when both the analytes and nucleic acid materials are present in aqueous solution.

The term “analyte” as referred to throughout the specification refers to any molecule or compound. A “volatile analyte” refers to a molecule or compound in gaseous or vapor phase, that is present, for example, in the headspace of a liquid, in ambient air, in a breath sample, in a gas, or as a contaminant in any of the foregoing. Analytes further include solid-phase compounds that are small enough to remain suspended in air, e.g., dust, molecules and compounds-present on the surfaces of particles present in gaseous or vapor phase, such as virus envelope proteins or bacterial cell surface or spore surface molecules, macromolecules that are cast off from other sources such as DNA, RNA, and proteins.

Accordingly, the present invention provides a nucleic acid-based chemical sensor, sensing system and sensing and identification method which provide for a nucleic acid-based multi-sensor, cross-reactive, sensor array having a rapid response time, a rapid sampling time, dynamic modulation of sampling and detection parameters, intelligent feedback control of analyte sampling conditions, smart mode sampling, smart detection through application of sophisticated analyte detection algorithms, high throughput screening of sensors, and high sensitivity, discrimination, and detection capability for a variety of target analytes.

The invention further provides a nucleic acid-fluorphore-based analyte sensing system which can transmit identifying information on various odors or smells, e.g., vapor or gaseous analytes, remotely, for example, over the Internet, or via a wireless communication system.

In one embodiment, the present invention provides a method for detecting and/or identifying an analyte, e.g., a volatile analyte, in an air sample comprising the steps of:
a) contacting said air sample with a nucleic acid-based sensor array comprising a substrate and a nucleic acid labeled with (attached to) a fluorophore dispersed on the substrate, said nucleic acid labeled with a fluorophore providing a characteristic optical response when subjected to excitation light energy in the presence of the analyte; and
b) detecting the presence or absence of the analyte.
c) identifying the analyte found in the air sample.

The substrate can be fabricated of different materials, including, for example, papers, fiberglass, silk, and fabrics made of synthetic materials.

In one preferred embodiment, the nucleic acid/fluorophore is dispersed on a plurality of internal and external surfaces within the substrate.

In one embodiment, contacting is accomplished by drawing an air sample suspected to contain the analyte into a sample chamber and exposing the array to the air sample. In a preferred embodiment, the air sample is drawn through the chamber for no more than five seconds.

The detecting may be accomplished by illuminating said sensor with excitation light energy and measuring an optical response produced by the sensor due to the presence of said volatile compound with a detector means. Detector means include, for example, a variety of photodetectors such as photomultiplier tubes (PMTs), charge-coupled display device (CCD) detectors, photovoltaic devices, phototransistors, and photodiodes. In a preferred embodiment, filtered photodiode detectors are used.

In all embodiments, the analyte can be identified by employing a pattern-matching algorithm and comparing the optical response of the nucleic acid-based sensor array with the characteristic optical response.

In specific embodiments, the analyte can be identified by measuring the spatio-temporal response patterns of the optical response and recognizing the patterns through a method selected from template matching, neural networks, delay line neural networks, or statistical analysis. The air sample may be suspected of containing analytes from a variety of substances, including explosive materials or chemical weapons agents.

The present invention flrther provides a sensing system for detecting and identifying an analyte in an ambient air sample. The system includes: a) a nucleic. acid-based sensor array comprising a plurality of nucleic acids; b) a fluorophore attached to the nucleic acids; c) a plurality of substrates wherein the nucleic acids with fluorophore are attached to; d) a substrate support; e) an excitation light source array including a plurality of light. sources optically coupled to the sensor elements; f) a detector array comprising a plurality of detectors optically coupled to said sensor elements; g) a sample chamber for housing the sensor elements, the light source array, and the detector array; h) a sampling means attached to the chamber for drawing the ambient air into the chamber for contact with the sensor array for a controlled exposure time; i) a controller means in electrical communication with the light sources, the detectors, and the sampling means, the controller means electrically coordinating and switching the sampling means with the light sources and the detectors for sampling the ambient air, measuring optical responses of the array sensors to the ambient air sample, and detecting the volatile compound; and j) an analyte identification algorithm for comparing the measured sensor optical responses to characteristic optical responses of the sensors to target analytes and identifying the analyte in the ambient air sample.

The elements of the analyte sensing system may be used together in a hand-held device, a device attached to another object, e.g., a shipping container, or used in conjunction with another screening device such as an x-ray screening machine. Alternatively, separate elements of the system, e.g., elements a)-i), can be used as one or more sensing units, while the analyte identification algorithm resides on a computer at a remote or separate location. One or more sensing units can be connected with the computer via a wired or wireless network.

In another preferred embodiment the identification algorithm reports a detection event when the sensor responses are different from blank air and identifies the analyte present using a pattern-match algorithm.

In one preferred embodiment, the system comprises one or more remote sensing units of the analyte sensing system with nucleic acid-fluorophore sensor arrays wirelessly connected to each others and the unit with the analyte identification algorithm, so that the information about the analytes is transferred to a remote location

Therefore, in one embodiment, the invention provides a sensor array sysiem for remote characterization of a gaseous or vapor sample, comprising: a) a plurality of sensors, wherein at least one sensor comprises nucleic acid/fluorophore combination comprising a plurality of nucleic acids attached to a fluorophore, wherein the plurality of sensors provide a detectable signal when contacted by an analyte; b) a measuring apparatus, in communication with plurality of sensors capable of measuring the detectable signal; c) a transmitting device, in communication with the measuring apparatus for transmitting information corresponding to the detectable signal to a remote location via the Internet, fiber optic cable, and/or an air-wave frequency; and a computer comprising a resident algorithm capable of characterizing the analyte.

The invention further provides a method of selecting nucleic acids capable of responding to a vapor phase analyte, said method comprising: a) contacting the nucleic acid labeled with a fluorophore with an analyte in vapor phase; and b) measuring the emission proflile of the fluorophore in the presence and absence of the target analyte, wherein a difference in the emission profile indicates that the nucleic acid is responsive to the analyte in vapor phase.

### BRIEF DESCRIPTION OF THE FIGURES

This invention is pointed out with particularity in the appended claims. Other features and benefits of the present invention can be more clearly understood with reference to the specification and the accompanying drawings in which:

FIGS. 1A and 1B show an example of a hand-held configuration of the Electo-Optical Vapor Interrogation Device (EVID). FIG. 1A shows a schematic view of the EVID sensor chamber, air flow path (30) (thick arrows), signal pathways (solid arrows), and computer control lines (dashed arrows) (8). The 3-way valve for switching between odorous and clean air is implemented as a pair of servo controlled valves (10). In FIGS. 1A and 1B the following parts are shown: panel of light emitting diode light sources (12) and excitation filters (28); the panel of photodiode detectors (26) and emission filters; (14); sniff pump (4); control and feedback control (double arrow in two directions) (16); computer (18); 16 channel integrating amplifiers and 20 bit A/D/ converters (20); inhale path (22) clean air from the source (24). FIG. 1B outlines a top view of the same system with LEDs (12); emission filter (14) excitation filter (28); and photodiodes (26).

FIGS. 2A and 2B show temporal responses of sensor made from YO-PRO and pBluescriptSK DNA. FIG. 2A shows a sensor made from YO-PRO, then rinsed in 70% ethanol for 5 min. FIG. 2B shows a sensor made from YO-PRO and 5 ng total pBlueScriptSK DNA. Analyte dilutions as fractions of saturated vapor were: Water, 10⁻¹; methanol (MeOH), 10⁻¹; triethylamine, 10⁻²; and propionic acid, 10⁻¹. Each trace represents the mean of 10 presentations; error bars indicate +/−1 S.D. For experiments with DNA-based analyte sensors, similar methods were used for each type of sensor. Briefly, DNA in solution was diluted to the desired concentration (0.2-40 ng/μl) in TE (1 Tris, 0.5 mM EDTA). 20 μl of dilute DNA was mixed with 1 μl concentrated dye stock and incubated at room temperature for 5 minutes. Dye-only controls were made of 1 μl dye stock in 20 μl TE. Sensors were made on a substrate of acid-washed 16xx silkscreen (10 mm×12 mm). DNA/dye mixtures were pipetted onto the substrate and allowed to dry for 25 minutes. Each sensor was rinsed in 70% ethanol for 5 minutes, allowed to dry, then attached to supports on glass coverslips for testing in the EVID (FIG. 2B).

FIGS. 3A and 3B show temporal responses of sensors made from different short sequences of single-stranded DNA and OliGreen dye. FIG. 3A shows an oligomer DS003, which has the sequence GATCCTTGCTACCCTCTCCTAGGAACGATGGGA (SEQ ID NO: 5). FIG. 3B shows an oligomer AJ001, which has the sequence ACCAGGACCTGACTAAGCAGAT (SEQ ID NO: 4). See FIG. 2 for sensor construction details and analyte dilutions. Each trace represents the mean of 10 replicates; error bars indicate +/−1 S.D.

FIGS. 4A and 4B show analyte concentration responses of two oligonucleotides labeled with the fluorescent dye Cy3(tm) during synthesis (using Cy3(tm) phosphoramidite from Glen Research). FIG. 4A shows LAPP1, which is the sequence GAGTCTGTGGAGGAGGTAGTC (SEQ ID NO: 1). FIG. 4B shows LAPP2, which is the sequence CTTCTGTCTTGATGTTTCTCAACC (SEQ ID NO.:2). The oligomers were stored in Tris-NaCl (10 mM Tris, 50 mM NaCl, pH 8) at 225 ng/μl, then diluted to a concentration of 50 ng/μl in distilled water just before use. See FIG. 2 for sensor construction details. Signal amplitudes are the parameters resulting from the exponential fit of the sensor temporal signals as described below. Sensor signals and data processing. Each data point is the mean of 10 presentations; error bars indicate +/−1 S.D.

FIG. 5 shows an overview of steps for sensor library creation and screening. The PCR template a) or primer extension template b) is amplified with two or one primer(s), respectively. Step 1: Synthesize random sequence library; Step 2: Dilute library; Step 3: Put samples into 104 96-well plates; Step 4: Amplify and label the nucleic acids; Step 5: Create high-density sensor library using a robotic spotter; Step 6: Image sensor library with array scanner before and after applying the vapor phase analytes.

FIG. 6 shows an example of a strong propionic acid odor response in one set of sensor spots. Data were collected using a ScanArray 4000 microarray scanner. The image on the left shows the background fluorescence of the sensor spots in clean air. The center image shows the fluorescence levels in the same spots after saturated vapor propionic acid was injected into the test chamber. The image on the right shows the change in fluorescence when the image on the left was substracted from the image in the middle. Arrays of spots were applied to the coverslip in blocks of 12×12 (12 replicates vertically and 12 different sequences horizontally); two replicate blocks (Rep 1 and Rep 2) were applied under three different ionic conditions: 50 MM MgCl₂, 50 mM NaCl, and water. One sensor sequence, TLAPP1 in water, showed a strong increase in fluorescence, other sequences showed smaller changes in fluorescence.

FIGS. 7A and 7B show a diagrams of an exemplary chamber (32) for delivering analytes to a sensor array when testing the nucleic acids for their responsiveness to analytes in vapor phase. FIG. 7A shows a top view of the chamber and FIG. 7B shows a side view of the chamber. Solid black (34) indicates stainless steel, darker grey (36) indicates 40 micron pore size stainless steel filter.

The tube wherein the odor is injected in is a 21 gauge Teflon tubing and is indicated with a white tube (38). The analyte is injected into the tube and comes out through the filter (40) (dark grey block). The interior chamber (42) contains the coverslip which is exposed to the analyte after the analyte is passed through the filter (white area inside the stainless steel walls of the chamber). The dimensions of chamber shown in this figure are appropriate for reading the glass coverslip with a ScanArray 4000.

FIG. 8 shows a block diagram of a sensor system of the present invention wherein the analysis is performed in a remote location showing the sensor chamber (1) with nucleic acid arrays (2) inside the chamber.

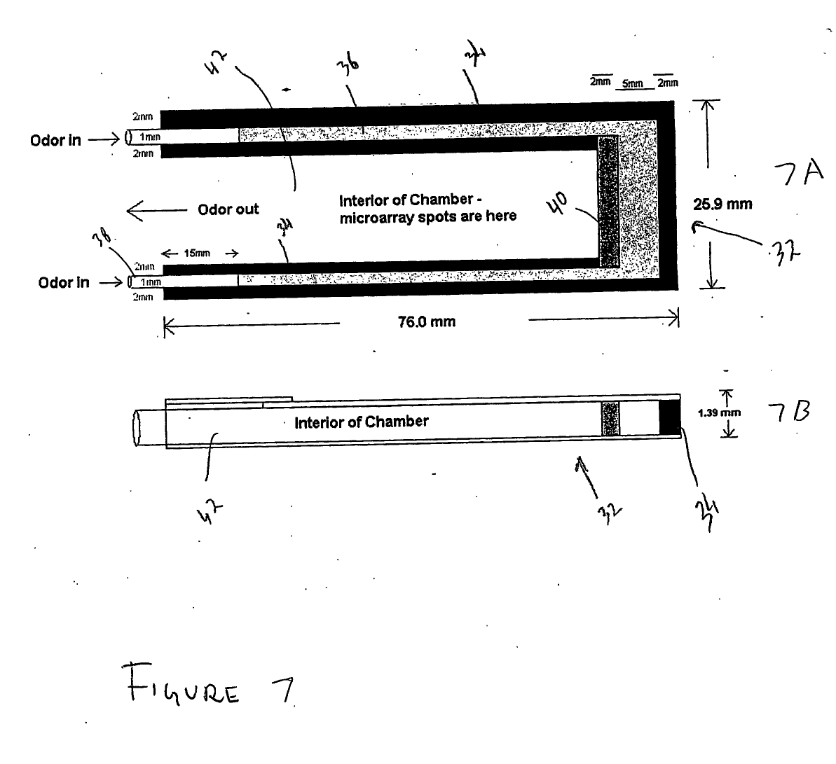

FIG. 9 is a block diagram showing hardware components of one embodiment of the system.

FIG. 10 is a schematic diagram of a sensor array module.

FIG. 11 is the sequence of oligomers with random internal sequence and fixed ends.

FIG. 12 shows PCR reaction primers and double stranded product. The asterisk represents Cy3(tm) labeling of the 5′ dTTP nucleotide of the lower primer.

### DETAILED DESCRIPTION OF THE INVENTION

The nucleic acid-based sensing method and sensing device design of the present invention mimics and parallels the structure and operational characteristics of the mammalian olfactory system through the combination of electro-optical hardware component modules, microprocessor control and software sampling and detection algorithms. The sample cavity design mimics the mammalian nasal cavity where odors or smells (i.e. vapor analytes) are drawn into the sensing module (“sniffed” or “inhaled”) and their interaction with a plurality of sensing elements (“sensory neurons”) in a sensor array triggers an external event.

Analyte applications include broad ranges of chemical classes such as organics including, for example, alkanes, alkenes, alkynes, dienes, alicyclic hydrocarbons, arenes, alcohols, ethers, ketones, aldehydes, carbonyls, carbanions, biogenic amines, thiols, polynuclear aromatics and derivatives of such organics, e.g., halide derivatives, etc., biomolecules such as sugars, isoprenes and isoprenoids, fatty acids and derivatives, etc.

Accordingly, commercial applications of the sensors, arrays and noses include environmental toxicology and remediation, biomedicine, materials quality control, food and agricultural products monitoring, anaesthetic detection, breath alcohol analyzers, hazardous spill identification, explosives detection, fugitive emission identification, medical diagnostics, fish freshness, detection and classification of bacteria and microorganisms both in vitro and in vivo for biomedical uses and medical diagnostic uses, monitoring heavy industrial manufacturing, ambient air monitoring, worker protection, emissions control, product quality testing, leak detection and identification, oil/gas petrochemical applications, combustible gas detection, H₂S monitoring, hazardous leak detection and identification, emergency response and law enforcement applications, illegal substance detection and identification, arson investigation, enclosed space surveying, utility and power applications, emissions monitoring, transformer fault detection, food/beverage/agriculture applications, freshness detection, fruit ripening control, fermentation process monitoring and control applications, flavor composition and identification, product quality and identification, refrigerant and fumigant detection, cosmetic/perfume/fragrance formulation, product quality testing, personal identification, chemical/plastics/pharmaceutical applications, leak detection, solvent recovery effectiveness, perimeter monitoring, product quality testing, hazardous waste site applications, fugitive emission detection and identification, leak detection and identification, perimeter monitoring, transportation, hazardous spill monitoring, refueling operations, shipping container inspection, diesel/gasoline/aviation fuel identification, building/residential natural gas detection, formaldehyde detection, smoke detection, fire detection, automatic ventilation control applications (cooking, smoking, etc.), air intake monitoring, hospital/medical anesthesia & sterilization gas detection, infectious disease detection and breath applications, body fluids analysis, pharmaceutical applications, drug discovery, telesurgery, and the like.

Biogenic amines such as putrescine, cadaverine, and spermine are formed and degraded as a result of normal metabolic activity in plants, animals and microorganisms and can be identified in order to assess the freshness of foodstuffs such as meats (Veciananogues, J. Agr. Food Chem., 45:2036-2041, 1997), cheeses, alcoholic beverages, and other fermented foods. Additionally, aniline and o-toluidine have been reported to be biomarkers for subjects having lung cancer (Preti et al., J. Chromat. Biomed. Appl. 432:1-11, 1988), breath ammonia in diagnosis, treatment assessment, and follow-up in hepatic encephalopathy (Shimamoto et al., Hepatogastroenterology, 47(32):443-5, 2000), while dimethylamine and trimethylamine have been reported to be the cause of the “fishy” uremic breath odor experienced by patients with renal failure. (Simenhoff, New England J. Med., 297:132-135, 1977). Thus, in general biogenic amines and thiols are biomarkers of bacteria, disease states, food freshness, and other odor-based conditions. Thus, the nucleic acid-fluorophore based nose sensor elements and arrays discussed herein can be used to monitor the components in the headspace of urine, blood, sweat, and saliva of human patients, as well as breath, to diagnose various states of health, such as the timing of estrus (Lane et al., J Dairy Sci 81(8):2145-50, 1998), and diseases as discussed herein. In addition, the sensor elements can be used for food quality monitoring, such as fish freshness (which involves volatile amine signatures), for. environmental and industrial applications (oil quality, water quality, air quality and contamination and leak detection), for other biomedical applications, for law enforcement applications (breathalayzers), for confined space monitoring (indoor air quality, filter breakthrough, etc.) and for other applications delineated above to add functionality and performance to sensor arrays through improvement in analyte detection by use in arrays that combine sensor modalities. Accordingly, the invention provides physicians and patients with a method to monitor illness and disease from remote locations. It is envisioned that the systems of the invention will be useful in medical care personnel monitoring patients who are bed-ridden at home or whom require continual monitoring of a particular disease state. Such remote monitoring ability eliminates the need for repeated trips to a doctors office or hospital and can provide physicians with real-time data regarding a patient's health and well-being.

In one embodiment, analyte interaction with the nucleic acid/fluorophore-based sensing elements produces emitted light energy at a detectable characteristic wavelength when the sensor elements are illuminated by excitation light energy from a filtered LED array. The multi-element nucleic acid-based sensor array of the present invention thus mimics the sensory neurons of the olfactory system in responding to the external triggering event, emitted light energy signaling the presence of an analyte, and detecting this triggering event by way of a filtered photodiode array (“Detection”). The photodiode preamplifiers mimic an olfactory sensory neuron by converting the optical signal to an electrical voltage signal (“Transduction”) which is amplified, manipulated and transported via electrical circuits (“Transmission”) to an analog-digital (“A/D”) converter and a software controlled microprocessor for data manipulation, analysis, feedback control, detection and identification (“Integration”). The Detection, Transduction, Transmission, and A/D features are replicated for each nucleic acid-based sensor element in the array. The sensor array of the present invention may be expanded or contracted without limit by adding or removing elements and channels according to the requisite analyte detection, discrimination and identification needs of a specific sampling application.

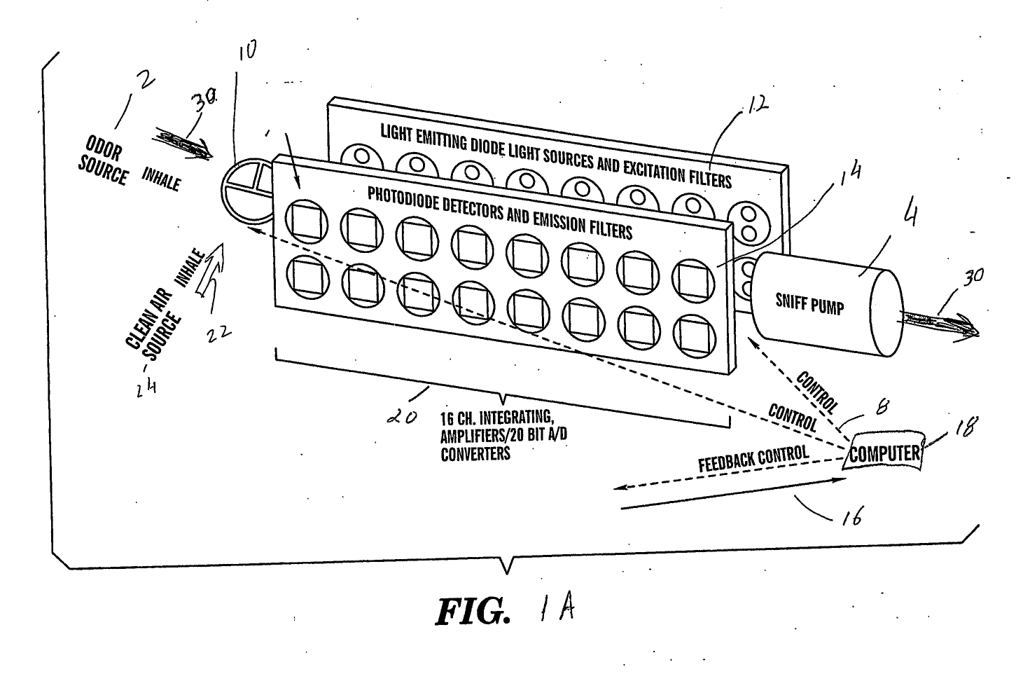

FIGS. 1A and 1B provide an overview of the analyte sensing and detection system of the present invention. Analytes from an odor source (2) are sniffed i.e. transported to the sensor array where the odors interact with the array of nucleic acid/fluorophore-based sensor elements using a “sniff pump” (4). Light energy excitation of the sensor elements (6) in the presence of the odors produces a detectable optical response signal due to changes in emitted light produced by analyte interaction with the nucleic acid/fluorophore compounds in the sensor elements. The spatio-temporal optical response of the nucleic acid-based array to the odor is detected, recorded, manipulated, and then matched to known target odors via smart analytical algorithms, resident at computer 18, which apply, for example, pattern matching, neural network, neuronal network, or statistical analysis methods to detect, discriminate and identify the odor.

The hardware and software components and configuration of the nucleic acid/fluorophore-based sensor of the present invention provide for a compact, portable, inexpensive, expandable, rapidly responding sensing device that can modify its detection strategy on the fly. The design and method provides for real-time, on-the-fly, modulation of: a) the output of light emitting diodes (LEDs), such as wavelength, intensity, and frequency; b) the detection properties of photodiodes, such as wavelength, gain, and frequency; c) the sampling parameters, such as frequency, duration, number, velocity, and rise-fall dynamics; and d) sampling time constant or temporal filter settings, for dynamically responsive, smart feedback control in sampling, detection and identification of analytes.

In addition to dynamic response modulation, the device and method further provide for hardware and algorithm implementations which evaluate the synchrony and noise characteristics across different sensors, especially those of the same composition being examined at different wavelengths. This provides a powerful tool for identifying and utilizing small response signals and rejecting noise.

By providing for independently illuminated, detected, recorded, and modulated sensing channels, levels of flexibility, expandability, portability, efficiency, and economy are achieved that are difficult to realize with the currently existing odor detectors. In addition, the use of small, inexpensive, flexibly programmable, computational microcomputer platforms and interchangeable nucleic acid/fluorophore-based sensors and sensor array modules provide for extreme flexibility and tailoring of sensor performance and capabilities to real world sensing applications, such as wirelessly connected sensing units. One or more of such units can be placed, for example, in a tunnel through which objects can be directed and screened for odors or analytes. Applications of such wireless systems comprising the nucleic acid-fluorophore sensor arrays of the present invention include, but are not limited to screening for mail, screening for trucks or ship containers, cars, luggage and other such subject for odors andlor analytes.

An example of a wireless system useful according to the present invention is described in detail in, for example, U.S. Pat. No. 6,631,333, incorporated herein by reference in its entirety.

Nucleic acids useful according to the present invention include single and double-stranded RNA and single and double-stranded DNA and cDNA. Nucleic acid, oligonucleotide, and similar terms used herein also include nucleic acid analogs, i.e. analogs having other than a phosphodiester backbone. For example, the so-called peptide nucleic acids, which are known in the art and have peptide bonds instead of phosphodiester bonds in the backbone are considered within the scope of the present invention (Nielsen et al. Science 254, 1497 (1991)). Alternatively, modified bases can be used in the nucleic acid sequence.

Examples of such modified bases are listed below on Table 1:

TABLE 1
Examples of Modified Bases

| Code | Modified base |
|---|---|
| ac4c | 4-acetylcytidine chm5u 5 |
| | (carboxyhydroxymethyl)uridine |
| cm | 2′-O-methylcytidine cm5u 5- |
| | carbamoylmethyluridine |
| cmnm | 5s2u 5-carboxymethylaminomethyl-2-thiouridine |
| cmnm | 5u 5-carboxymethylaminomethyluridine |
| d | dihydrouridine |
| fm | 2′-O-methylpseudouridine |
| gal | q beta,D-galactosylqueuosine |
| gm | 2′-O-methylguanosine i-inosine |
| i6a. | N6-isopentenyladenosine |
| m1a | 1-methyladenosine |
| m1am | 2′-O-methyl-1-methyladenosine |
| m1f | 1-methylpseudouridine |
| m1g | 1-methylguanosine |
| m1i | 1-methylinosine |
| m22g | 2,2-dimethylguanosine |
| m22gm | N2,N2,3′-trimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| mam5s2u | 5-methylaminomethyl-2-thiouridine |
| mam5u | 5-methylaminomethyluridine |
| man q | beta,D-mannosylqueuosine |
| mcm5s2u | 5-methoxycarbonylmethyl-2-thiouridine |
| mcm5u | 5-methoxycarbonylmethyluridine |
| mo5u | 5-methoxyuridine |
| ms2i6a | 2-methylthio-N6-isopentenyladenosine |
| ms2t6a | N-((9-beta-D-ribofuranosyl-2-methylthiopurin-6- |
| | yl)carbamoyl) threonine |
| mt6a | N-((9-beta-D-ribofuranosylpurine-6-yl)N-methyl- |
| | carbamoyl) threonine |
| mv | uridine-5-oxoacetic acid methylester |
| o5u | uridine-5-oxyacetic acid(v) |
| osyw | wybutoxosine |
| p | pseudouridine |
| q | queuosine |
| s2c | 2-thiocytidine |
| s2t | 5-methyl-2-thiouridine |
| s2u | 2-thiouridine |
| s4u | 4-thiouridine |
| t | 5-methyluridine |
| t6a | N-((9-beta-D-ribofuranosylpurine-6- |
| | yl)carbamoyl)threonine |
| tm | 2′-O-methyl-5-methyluridine |
| um | 2′-O-methyluridine × 3-(3-amino-3- |
| | carboxypropyl)uridine,(acp3)U |
| yw | wybutosine |

The length of the nucleic acid sequences can vary between about 1 base of single stranded DNA up to about 3 thousand bases of double stranded DNA. Preferably about 18-24 base pair oligonucleotides are used.

Nucleic acids useful according to the present invention can be synthesized using methods well known to one skilled in the art. For example, a solid-phase phosphotriester approach can be used as described in Sproat et al. (Solid-phase synthesis of oligodeoxyribonucleotides by the phosphotriester method, in Oligonucleotide Synthesis—A practical approach (Gait, M. J., Ed.), IRL Press, Oxford pp. 83-115, 1984). The concept of the solid-phase phosphotriester approach has four basic aspects: the oligonucleotide is synthesized while attached covalently to a solid support, excess soluble protected nucleotides and coupling reagent can drive a reaction near to completion, the reaction is carried out in a single reaction vessel to diminish mechanical losses due to solid support manipulation, allowing synthesis with minute quantities of starting materials, and the heterogeneous reactions are standardized. All these procedures are easily automated and several commercially available oligonucleotide synthesizers are known to one skilled in the art. The most used chemical route for solid-phase oligonucleotide synthesis is the phosphite triester method as modified by Beaucage and Caruthers (Beaucage, S. L., and Caruthers, M. H. (1981) Deoxynucleoside phosphoramidites—A new class of key intermediates for deoxypolynucleotide synthesis, Tetrahedron Lett. 22,1859-1862).

Alternatively, nucleic acids can be isolated from libraries comprising nucleic acid fragments in the form of, for example, plasmids, cosmids, yeast artificial chromosomes, and bacterial artificial chromosomes. The nucleic acids can also be isolated from any other source such as viruses, and procaryotic or eucaryotic cells. Nucleic acid isolation methods are routine and protocols can be found, for example from Molecular Cloning: A Laboratory Manual, 3rd Ed., Sambrook and Russel, Cold Spring Harbor Laboratory Press, 2001.

After isolation, the isolated nucleic acids can be further modified, for example, by restriction enzyme digestion. Isolated nucleic acids can also be amplified using PCR and either random or specific primer sequences. Such primer sequences can also be labeled with a fluorophore during the oligonucleotide synthesis.

In one embodiment, the nucleic acids useful in the present invention are similar to aptamers which can be selected from existing aptamers or from random sequence libraries. Aptamers are defined as single-stranded or double-stranded nucleic acids which are capable of binding proteins or other small molecules with high specificity in aqueous solution. In the present invention, the nucleic acid-based sensors differ from aptamers in two important respects: 1) the nucleic acids sensors are dried onto a substrate and interact directly with compounds in the air, and 2) the nucleic acid sequences are selected for their capacity to react, in combination with a fluorophore, to broad ranges of volatile compounds. Generally, aptamers are selected from a large number of non-interacting oligonucleotides and they originate from in vitro selection experiments termed “SELEX” for systematic evolution of ligands by exponential enrichment, that, starting from random sequence libraries, optimize the nucleic acids for high-affinity binding to given ligands (C. Tuerk and L. Gold, Science 249, 505 (1990); A. D. Ellington and J. W. Szostak, Nature 346, 818 (1990)). Reviews on in vitro selection and aptamers, see, e.g., G. F. Joyce, Curr. Opin. Struct. Biol. 4, 331 (1994); L. Gold, B. Polisky, O. C. Uhlenbeck, M. Yarus, Annu. Rev. Biochem. 64, 763 (1995); J. R. Lorsch and J. W. Szostak, Acc. Chem. Res. 29, 103 (1996); T. Pan, Curr. Opin. Chem. Biol. 1, 17 (1997); Y. Li and R. R. Breaker, Curr. Opin. Struct. Biol. 9, 31.5 (1999); M. Famulok, Curr. Opin. Struct. Biol. 9, 324 (1999).

The length and nucleic acid sequence can be easily modified and thereby the repertoire of possible sensors is almost infinite raging from preferably about 1 to about 3000 bases. The preferred lengths of the sensing nucleic acid sequences vary from about 10 to about 2000 bases, from about 10 to about 1000 bases, from about 10 to about 500, from about 15 to about 400 bases, from about 15 to about 300 bases, from about 15 to about 200 bases, from about 15 to about 100 bases, from about 15 to about 30, 40, 50, 60, 70, 80, or 90 bases. In a preferred embodiment, the nucleic acids are about 15-24 bases long.

To test the responsiveness of the nucleic acid to an analyte, the isolated or synthesized nucleic acids are labeled with a fluorophore. As used herein the term “fluorophore” will be understood to refer to both fluorophores, phosphors and luminophores, and to chromophores that absorb but do not emit photons.

Fluorophores provide the means for transducing the interaction of the analytes with the sensor. For example, fluorophores useful according to the present invention include, but are not limited to OLIGREEN (Molecular Probes, Inc., Eugene, Oreg.), and other fluorescent dyes listed in the following Table 2.

TABLE 2
Examples of Fluorophores and Their Excitation and Emission
wave-lenghts.

| Fluorescent Dye | Excitation, nm | Emission, nm |
|---|---|---|
| 5-FAM | 494 | 518 |
| Alexa ™ 488 | 495 | 520 |
| Alexa ™ 532 | 531 | 554 |
| Alexa ™ 546 | 555 | 570 |
| Alexa ™ 555 | 555 | 565 |
| Alexa ™ 568 | 579 | 604 |
| Alexa ™ 594 | 590 | 615 |
| Alexa ™ 647 | 649 | 666 |
| Alexa ™ 647 | 649 | 666 |
| Alexa ™ 660 | 663 | 690 |
| Alexa ™ 660 | 663 | 690 |
| Allophycocyanin (APC) | 650 | 660 |
| Allophycocyanin (APC) | 650 | 660 |
| BODIPY ® 564/570 | 564 | 570 |
| BODIPY ® TMR | 542 | 574 |
| BODIPY ® 530/550 | 530 | 550 |
| BODIPY ® 558/568 | 558 | 568 |
| BODIPY ® 630-650 | 630 | 650 |
| BODIPY ® 630-650 | 630 | 650 |
| Calcein | 494 | 517 |
| Calcium Crimson ™ | 590 | 615 |
| Calcium Green ™ | 506 | 533 |
| Calcium Orange ™ | 549 | 576 |
| C-Phycocyanin | 620 | 648 |
| Cy2 ™ | 489 | 506 |
| Cy3.5 ™ | 581 | 596 |
| Cy3 ™ | 550 | 570 |
| Cy5.5 | 675 | 694 |
| Cy5.5 | 675 | 694 |
| Cy5 ™ | 649 | 670 |
| Cy5 ™ | 649 | 670 |
| DiD DilC(5) | 644 | 665 |
| DiD DilC(5) | 644 | 665 |
| dsRed | 558 | 583 |
| Ethidium Bromide | 518 | 605 |
| FAM | 488 | 508 |
| FITC | 494 | 518 |
| FluorX ™ | 494 | 519 |
| GFP | 488 | 558 |
| GFP Red Shifted | 488 | 507 |
| (rsGFP) | | |
| JOE | 522 | 555 |
| JOE-514 | 514 | 549 |
| Magnesium | 506 | 531 |
| Green ™ | | |
| Magnesium | 550 | 575 |
| Orange ™ | | |
| Nile Red | 549 | 599 |
| Oregon Green ™ | 496 | 524 |
| 488 | | |
| Oregon Green ™ | 503 | 522 |
| 500 | | |
| PBXL-1 | 545 | 666 |
| PBXL-3 | 614 | 662 |
| Phycoerythrin, R & B | 565 | 575 |
| Pyronin Y | 555 | 580 |
| Red Reflect | 633 | 633 |
| Red Reflect | 633 | 633 |
| Rhodamine 110 | 496 | 520 |
| Rhodamine 123 | 507 | 529 |
| Rhodamine B | 555 | 580 |
| Rhodamine Green ™ | 502 | 527 |
| Rhodamine | 542 | 565 |
| Phalloidin | | |
| Rhodamine Red ™ | 570 | 590 |
| RiboGreen ™ | 500 | 525 |
| ROX | 580 | 605 |
| R-phycocyanin | 618 | 642 |
| R-Phycoerythrin (R- | 565 | 575 |
| PE) | | |
| SYBR Green | 497 | 520 |
| Sypro Ruby | 450 | 610 |
| TAMRA | 555 | 575 |
| Thiadicarbocyanine | 651 | 671 |
| Thiadicarbocyanine | 651 | 671 |
| TO-PRO ™-1 | 514 | 533 |
| TO-PRO ™-3 | 642 | 660 |
| TO-PRO ™-3 | 642 | 660 |
| YO-PRO ™-1 | 491 | 509 |
| YO-PRO ™-3 | 612 | 631 |
| YOYO ™-3 | 612 | 631 |

Preferred dyes useful according to the present invention include OLIGREEN or YO-PRO dye (Molecular Probes, Inc., Eugene, Oreg.).

In addition to labeling each oligonucleotide with a single type of fluorophore, fluorophore/quencher systems can also be used. Typically, these systems incorporate a fluorophore (e.g., fluorescein) and a quencher (e.g., DABCYL) at the ends of an oligomer sequence that forms a hairpin structure (see, e.g., Tyagi, S. and Kramer, F. R. (1996) Nature Biotech., 14:303-308; Hamaguchi, N., Ellington, A., and Stanton, M. (2001) Anal. Biochem., 294:126-131). In this conformation, the DABCYL quenches the fluorescein fluorescence through fluorescence resonance energy transfer (FRET). Upon binding of the oligomer sequence to its target ligand, the conformation of the oligomer changes, separating the fluorophore and quencher. This separation decreases the FRET between the fluorophore and quencher, causing a change in fluorescence at the fluorophore emission wavelength.

These energy transfer pairs for fluorophore/quencher systems where both the donor and acceptor are covalently bound to the same nucleic acid are known to one skilled in the art. Such energy transfer pairs have been used to detect changes in oligonucleotide conformation, such as in Tyagi et al. (EP 0 745 690 A2 (1996)) and Pitner et al. (U.S. Pat. No. 5,691,145 (1997)). They also have been used to detect cleavage of the oligonucleotide at a point between the donor and acceptor dyes, such as in Han et al. (U.S. Pat. No. 5,763,181 (1998)), Nadeau et al. (U.S. Pat. No. 5,846,726 (1998)), and Wang et al. (ANTIVIRAL CHEMISTRY & CHEMOTHERAPY 8, 303 (1997)). Energy transfer pairs covalently bound to oligonucleotides have also been used to provide a shift in the ultimate emission wavelength upon excitation of the donor dye, such as by Ju (U.S. Pat. No. 5,804,386 (1998)).

Other fluorophore/quencher systems have been described in the art and such systems can be used according to the present invention. For example, the combination of a non-covalently bound nucleic acid stain with a covalently attached fluorophore on a single-stranded oligonucleotide hybridization probe has been used to detect specific DNA target sequences by monitoring the fluorescence of either the nucleic acid stain or the covalent label, such as described in Lee and Fuerst (PCT Int. Appl. WO 99 28,500). Also, U.S. Pat. No. 6,333,327 discloses fluorophore/quencher systems for decreasing background fluorescence during amplification assays and in ligation assays, and for detecting hybridization.

The nucleic acid can be labeled with the fluorophore at the 3′ region, 5′ region of the nucleic acid, or internally. Additionally, the flurophore can be applied dye.

Nucleic acids in the nucleic acid-based sensors of the present invention are labeled using techniques known to one skilled in the art. Such methods include, for example, mixing the nucleic acids with a dye, end-labeling the nucleic acids during oligonucleotide synthesis, or labeling the nucleic acids during a PCR reaction. According to the present invention, any method to attach the fluorophore to the nucleic acid can be used.

Preferred examples of applying or using dyes to label nucleic acids include direct application of dyes, such as for example OLIGREEN and TOTO family of cyanine dimer dyes (Molecular Probes, Inc.), onto the nucleic acids to produce a labeled nucleic acid.

Nucleic acids can also be labeled during their synthesis. Reagents are readily available (e.g., Glen Research, Sterling, Va.) for adding fluorescent dye molecules to the 3′ and 5′ ends, as well as labeled dT for inserting the dye molecule within the nucleic acid sequence. Use of direct labeling allows control over the precise amount and location of the fluorophore within the nucleic acid sequence. Also, a fluorophore may be added at different locations or multiple fluorophores at several locations in the nucleic acid sequence which allows development of even greater variety of sensors.

The sequence and/or structure of the nucleic acid used to construct a sensor effects the response profile of the sensor. In preparing the nucleic acid-based sensors, the effect of sequence (and, hence, structure) on the response properties of nucleic acid-based sensors is tested. For each sequence tested, the folding structure(s) and melting temperature(s) are estimated to determine the effect of a specific DNA structure on the analyte responses.

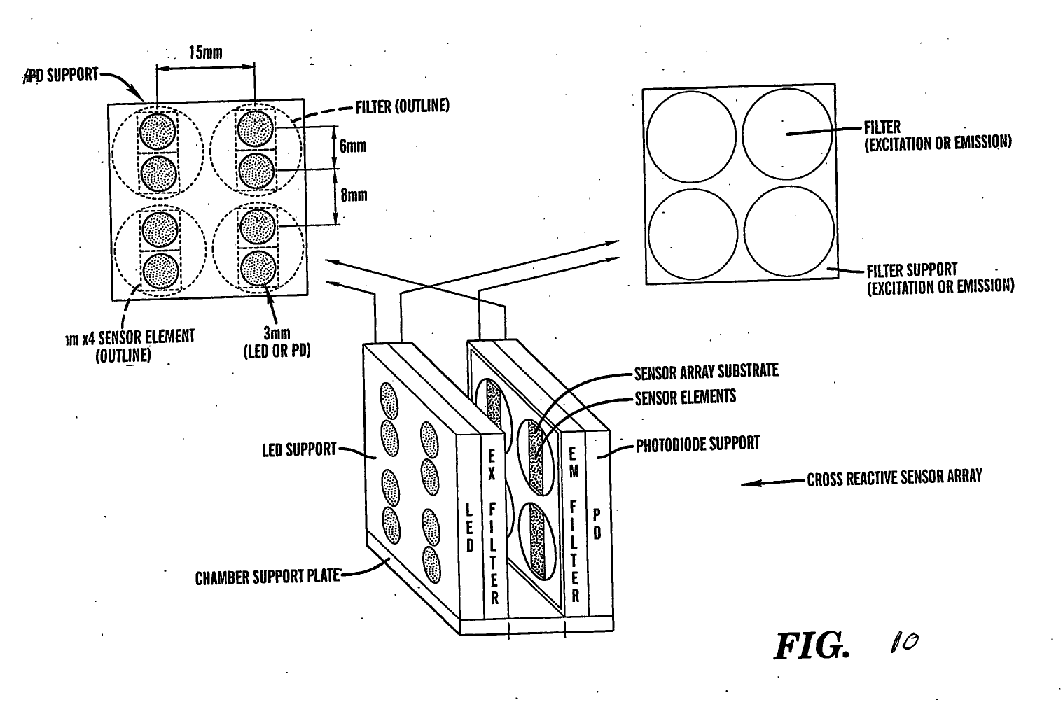

The amount of nucleic acids used in producing the nucleic acid-based sensor effects the response of the sensor to an analyte. For example, effects of DNA quantity were seen in preliminary experiments on the nucleic acid based sensors (FIG. 11). Therefore, for each sensor configuration, a range of nucleic acid and dye concentrations (for applied dyes) are tested for the amount that produces the desired result, i.e. a clearly noticeable response to a test analyte.

It is desirable to apply the nucleic acid/fluorophore solution to the substrate as evenly as possible. For example, an inkjet application system can be used. With this system, a piezo-electric inkjet ejects 50 nl droplets of solution, which are applied to the substrate in precise locations using an XYZ positioning system. The inkjet system can be used to apply nucleic acid/fluorophore solutions to the sensor substrates.

The substrate used to make the sensor can be fabricated of different materials, such as, for example, papers, fiberglass, fabrics made of synthetic materials. However, for the purpose of screening/testing nucleic acid/fluorophore combinations for their responsiveness to test analytes, a glass substrate can be used. The nucleic acid/fluorophore combination should then be tested on the substrate that is intended to be used in the sensor to ensure responsiveness will not be effected.

Long-term stability of the nucleic acid/fluorophore -based sensor responses is important for their use in the present invention. Fluorescent dyes can photobleach upon repeated exposure to excitation light, and different dyes photobleach at different rates. The present invention is designed to minimize photobleaching (by limiting light exposure to brief 1 msec pulses), and the analyte recognition algorithms are resistant to changes in signal amplitude. Reducing any possible photobleaching, however, will increase the life expectancy of the sensor.

Dried nucleic acids are stable for long periods of time which makes it an ideal sensor material. However, it is possible that the nucleic acid used in the analyte sensors degrades over time thereby altering analyte response. The degradation is likely to be minimal and can be easily tested. Analyte responses over repeated sniffs are compared to the data from, for example the photobleaching tests described above. Any signal decrease that cannot be accounted for by photobleaching will suggest a nucleic acid degradation effect. If evidence of nucleic acid degradation is found, nucleotide modifications that reduce nuclease degradation can be used to reduce degradation as described, e.g., for applications to aptamers (see Jayasena, S. D. (1999). Clin. Chem., 45:1628-1650).

The present invention also provides a system for identifying and selecting nucleic acid-fluorophore combinations for their capacity to respond to odors and/or vapor phase analytes. The method includes exposing the nucleic acids-fluorophore complexes to an analyte in a vapor phase and comparing the emission of light from the complex before addition to the analyte and during or after exposure to the analyte, wherein difference in the emitted light from the fluorophore between the before and during or after exposure to the analyte indicates that the nucleic acid-fluorophore is capable of responding to the analyte.

For example, a plurality of different nucleic acid-fluorophore complexes can be applied onto a substrate, such as a glass coverslip, the substrate is then placed in the array scanner and scanned to produce the “before” image. While the chamber is still in the scanner, an analyte in vapor phase is injected into it using, for example a syringe. For example, a syringe can be used to withdraw some of the headspace vapor. from a container containing a sample analyte. The amount of analyte can vary depending on the size of the chamber and can be as little as about 0.25-0.5 ml or about 1, 2, 3, 5, 10, 15 or up to 100 ml. In the preferred embodiment, about 0.5-2.5 ml of vapor analyte is added to the test chamber.

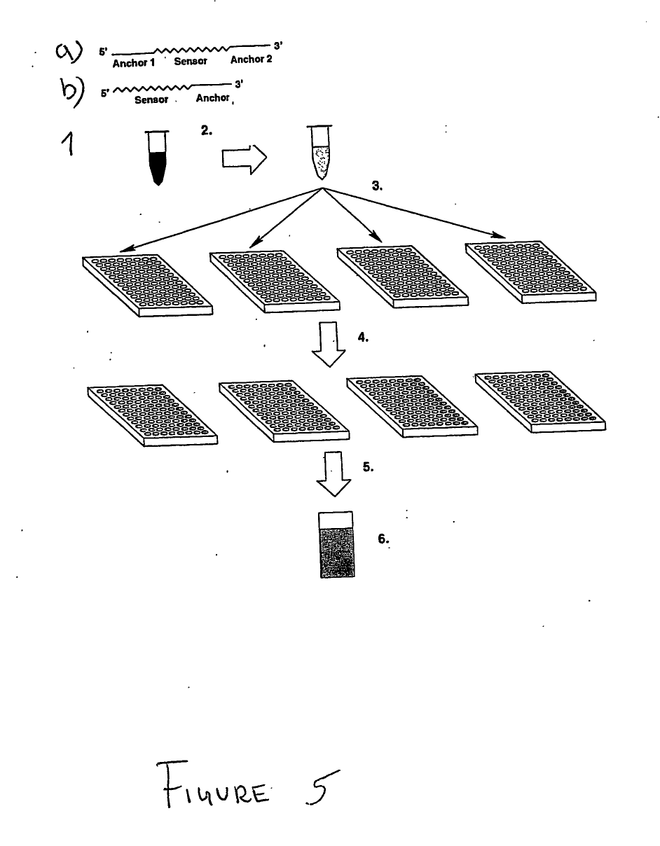

The exposure time may be varied from about 1 second to up to 5, 10, 15, 20, 25, 30, 40 and 50 seconds and further up to several minutes, for example, 1, 2, 3, 4, 5 and up to 10 minutes. Most preferably, the exposure time varies between about 25 seconds to about 3 minutes. After injection of the test analyte odor to the chamber with the substrate, the chamber is scanned again to get the “after” image. If there is a difference between the before and after images, in any of the particular coordinates with a nucleic acid-fluorophore complex, the complex is considered reactive to that particular test analyte in vapor phase. The difference between the before and after image may be any detectable difference in the intensity of emission light between the before and after image. FIG. 6 shows an example of a screen for nucleic acids in a microarray form, wherein difference of light emission pattern can clearly be appreciated.

The exposure time and test analyte vapor amount may vary because the goal of using the chamber and scanner is to find any and all nucleic acid-fluorophore spots that respond to the test analytes at all. The main motivations for the times and volumes is to make sure all spots are covered by the analyte vapor. In one embodiment, a 2 ml chamber is used and about volumes of 2-10 rnl of vapor analyte can be injected into the chamber. With high concentration vapor (i.e., saturated vapor), smaller volumes may be sufficient. With low concentrations, higher volumes are needed to make sure the air in the chamber is sufficiently exchanged.

The gas or vapor phase analyte is preferably injected into the chamber relatively slowly. For example, for the about 2 ml chamber, ihe injection speed is most preferably about 0.5-1 ml/sec.

The present invention further provides nucleic acid/fluorophore-based array sensor element compositions disposed on substrates which may be either inert or active during analyte sampling and detection. While inert supports are typically used in conventional sensing devices, the present invention provides for active dye support materials that enhance sensor responses to specific analytes by their unique chemical; physical, adsorption, or optical characteristics. Different substrate support materials may be employed within a single array where specific support materials are matched to specific fluorophores, fluorophore compounds and nucleic acid/fluorophore mixtures to produce enhanced sensor responses to specific volatile analytes or odors.

Fibrous substrate supports, which enhance sensor response signals for a variety of fluorophores and nucleic acid/fluorophore mixtures are preferred substrate materials.

An additional advantageous feature of the present invention is in providing for removable or interchangeable nucleic acid/fluorophore -based arrays, array substrates, or substrate supports to facilitate changing sensor arrays to match specific analyte sampling and detection requirements. In one embodiment, multiple layers of array substrates may be employed for diversification and enhancement of sensor detection capabilities for identifying both broad and specific classes of analytes.

One skilled in the art would recognize that it is generally preferred to position sensor substrates at the appropriate viewing angle and distance from light emitting diode excitation light sources and photodiode detectors so as to provide for optimum sensor signal generation and detection. In one preferred embodiment, a separate substrate holder may be provided for positioning and securing array substrates., In an alternative preferred embodiment, the sample chamber housing may be configured for proper positioning and securing array substrate.

As will be appreciated by those in the art, the. number of possible substrate materials is very large. Possible substrate materials include, but are not limited to, silk, glass and modified or functionalized glass, plastics (including acrylics, polystyrene and copolymers of styrene and other materials, polypropylene, polyethylene, polybutylene, polyurethanes, teflons, etc.), polysaccharides, nylon or nitrocellulose, resins, silica or silica-based materials including silicon and modified silicon, carbon, metals, inorganic glasses, plastics, and a variety of other polymers.

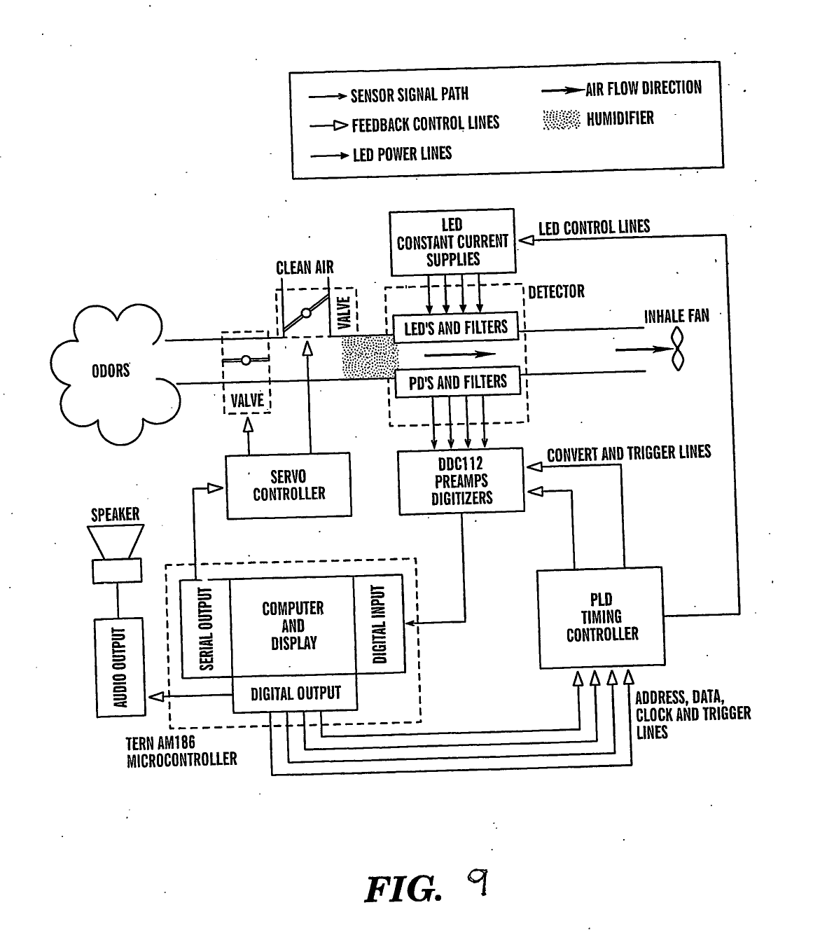

In preferred embodiments, optically transparent substrates are employed to permit placement of the substrate between LED light sources and photodiode detectors as shown in FIG. 10. In alternative embodiments, where the LEDs and photodiodes are placed on the same side of the substrate, optically opaque or optically absorbing, reflective, and scattering materials may be employed.

Where conventional flat, planar, curved or non-planar solid sensor substrates are used, these substrates are generally self-supporting and substrate supports are not required but may be optionally employed.

While conventional flat, planar, or curved non-planar solid sensor substrates may be employed, increased sensor surface area can arise from depositing dyes on highly convoluted surfaces that include fine fibrous hairs of different materials, particulates, porous substrates, or films and substrates suspended within the sampling stream. With the innovative substrates of the present invention, these preferred substrate embodiments provide enhanced contact and interaction between sample target analytes and sensor elements, increased optical response signal per unit of sensor geometrical surface area, and increased optical response signal per unit of sensor volume.

In preferred embodiments, highly permeable, high surface area, textured, fibrous or particulate substrates which have substantial open porosity for unimpeded transport of vapors and fluids are desired. In preferred embodiments, single or multiply layers of papers, felts, laid, or woven fibrous materials or fabrics are employed. In alternative embodiments, loosely packed individual fibrous or particulate materials may be employed.

In a most preferred embodiment, fibrous substrate materials are employed for signal enhancement. Important considerations in selecting fibrous substrates are substrate permeability to vapors, high accessible surface area per unit volume, response signal enhancement for specific analytes, how the substrate interacts with the sample flow to provide open access of its external and internal surfaces to analytes for interaction with the sensing material. While particularly useful fiber substrates are porous, lightweight paper or tissue products, for example Kimwipe™ (Kimberly-Clark Corp., Roswell, Ga.), lens papers, facial tissues, and products made from cotton, rayon, glass, and nitrocellulose fibers, other fibrous materials employing natural or synthetic fibers such as felt, batting, textiles, woven fabrics, yarns, threads, string, rope, papers, and laminates or composites of such materials would be equally suitable as long as they possess the requisite fluid permeability, surface area, surface area to volume ratio, and open porosity for free transport of vapor and fluid analytes.

Particularly useful inorganic fibers and fibrous material compositions are natural and synthetic fibers made from glass, ceramic, metal, quartz, silica, silicon, silicate, silicide, silicon carbide, silicon nitride, alumina, aluminate, aluminide, carbon, graphite, boron, borate, boride, and boron nitride. Particularly useful natural or synthetic fibers and fibrous material compositions are polymer fibers made from aromatic polyamides, nylons, polyarylonitrile, polyesters, olefins, acrylics, cellulose, acetates, anidex, aramids, azlon, alatoesters, lyocell, spandex, melamines, modacrylic, nitrile, polybenzinidazole, polyproplylene, rayons, lyorell, sarans, vinyon, triacetate, vinyl, rayon, carbon pitch, epoxies, silicones, sol gels, polyphenylene-benzobis-ozazole, polyphenylene sulfides, polytetrafluoroethylene, teflon, and low density or high density polyethylene. In one preferred embodiment, fiber materials that are highly absorbent and have good dye retention characteristics, for example the cellulosic fiber known as Lyorell, may be employed.

In alternative embodiments, fibers may be coated with either chemical sizing, polymer, ceramic or metallic materials. Chemical sizing such as modified polyvinyl acetates, organosilanes, coupling agents, anti-static agents and lubricants may be employed as appropriate.

With respect to signal enhancing sensor substrate properties of the present invention, one skilled in the art would generally recognize and understand the intended meaning of the term “textured” referring to material surfaces which typically have a distribution of surface topographical features, such as high points (peaks) and low points (valleys), ranging from about 100 nm to about 1000 μm RMS. The term “high permeability” generally refers to materials and material structures with a high open porosity that provide essentially free, unimpeded access and convective or diffusive transport to, low viscosity fluids, the term “high surface area” generally referring to materials that have a surface area of at least 1 M²/g and typically refers to surface areas ranging between 2 to 500 M²/g. The term “high surface area to volume” generally refers to materials having a surface area to volume ratio of at least 1M²/cm³, and typically refers to surface area to volume rations ranging between 2 to 1000 1M²/cm³. The terms “porous” or “porosity” generally referring to materials having a distribution of pore sizes ranging from 100 nm to 1000 μm, and the term “high open porosity” generally referring to materials whose pore distributions substantially comprise open pores.

In alternative embodiments, the sensor substrates of the present invention may be chemically or physically modified to enhance surface area, absorption, adhesion, hydrophobicity, hydrophilicity, repulsion, discrimination or specificity. In some embodiments, the substrate may be chemically altered to provide chemical functionality for interaction with analytes, such as providing for enhanced affinity, enhanced repulsion, or steric impediments to analyte adsorption.

In a preferred embodiment, the sensors are made on a substrate of acid-washed silkscreen, preferably 16xx and sized about 10 mm×12 mm. The nucleic acid/fluorophore mixture is pipetted onto a silkscreen, preferably about 5-50 μl of nucleic acid/fluorophore mixture is used, and allowed to air dry for about 10-60 minutes, preferably about 20-30 minutes, most preferably about 25 minutes. Each sensor is rinsed in 70% ethanol for about 5 minutes, allowed to air dry, then attached to supports on, for example, glass coverslips.

The nucleic acid/fluorophore-based sensor and sensing system of the present invention provides for a rapidly responding, relatively inexpensive, dynamically configurable, intelligent, portable sampling device.

One preferred detection devise useful with the nucleic acid-fluorophore. sensors of the present system is described in detail in the issued U.S. Pat. No. 6,649,416, which is herein incorporated by reference in its entirety.

The device delivers analytes (odors) in a controlled, pulsatile manner (sniff) to nucleic acid/fluorophore-based sensor array and detector array system that generates signals, for example, analog electrical signals. The number of sensors, detectors, and sampling time points can be made larger or smaller depending on the classes of analytes that are being targeted for detection. Analog signals, for example, are amplified and filtered by a pre-amplifier/amplifier module and digitized by an analog/digital conversion module for storage in a computer memory module. All attributes of the sensing process, including odor delivery, sampling, analysis, detection and identification are under programmable software control via a computer.

The sensing device housing the nucleic acid/fluorophore-based array is easily trained to recognize specific analytes. Training consists of delivering a known set of analytes, for example DNT and other nitroaromatic compounds for detection of explosives, to the device, one analyte at a time, and storing matrices of values that are spatio-temporal signatures of each analyte in memory. When an unknown analyte is to be sampled after training, it is delivered to the device and a matrix of values acquired from the unknown is compared to matrix templates for the variety of analytes stored in memory during the training phase. The best match between the unknown and the library of stored matrices is then determined using a number of different algorithms. In one embodiment, the algorithm looks for the best match after calculating the sum of the squared differences between each point in the stored and unknown matrices. In a preferred embodiment, the rising phase of each sensor signal is fit by an exponential function containing two parameters describing the signal amplitude and rate of change. A matrix of these parameters is then used to represent the sensor array response, and matches are calculated as above using sum of squared differences.

The sensing system provides output results in a variety formats including, but not limited to screen displays, plots, printouts, database files, and recorded or synthesized voice messages.

The sensing device of the present invention comprises a sampling chamber housing an analyte delivery system and a multi-channel array comprising light emitting diodes (LEDs) focused through an array of excitation filters onto individual sensor elements of a sensor array. An array of photodiodes, filtered with an array of emission filters, detects emitted light energy produced by illuminating the sensor elements with LED excitation light during interaction with analytes that are drawn into the sample chamber by the analyte delivery system. The ambient temperature, humidity, and particulate levels in the sample chamber may be controlled for improved reproducibility in sampling under a variety of environmental conditions.

The sensing device generally provides the basic function comprising analyte delivery and control (i.e. manipulation of spatial and temporal distributions; control over temperature, humidity, and duty cycle), detection by a sensor array and transduction of sensor signals into a manipulatable format, analysis of transduction output events, and dynamic feedback control over analyte delivery, detection and analysis for intelligent sampling and detection and optimization of nucleic acid/fluorophore-based sensor sensitivity and analyte discrimination.

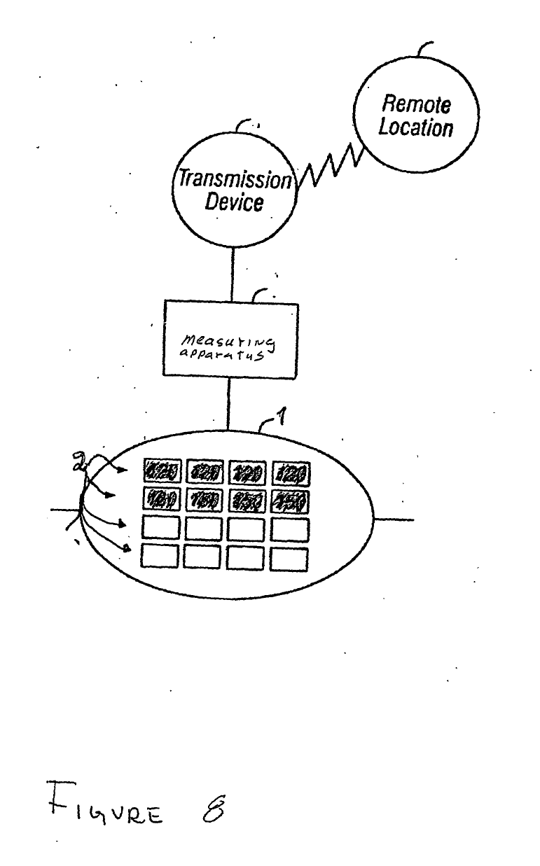

FIG. 9 provides a schematic block diagram showing the general modular design aid configuration of the preferred nucleic acid/fluorophore -based sensor array and sensing system components. A detailed schematic of an exemplary sensor array configuration showing LEDs, excitation filters, sensor elements, (nucleic. acid/fluorophore) sensor array substrates, emission filters, and photodiodes is provided in FIG. 1B.

In a preferred embodiment, the analyte delivery system provides feedback control over sample temperature, humidity, flow-rate, and the rise and fall times, duration, and frequency of analyte delivery.

Generally, the sensing chamber includes: a) a means for controlling temperature, humidity, air flowrate, rise and fall times and frequency of the applied vapor pulses; b) a means for controlling the surface properties of the sensing and non-sensing areas of the chamber (liquid, mucus, or gel lining) in order to impart chromatographic surfaces to the sensing area and/or humidify, dehumidify, or distribute the analyte to the sensory surface, or to optimize response of the sensing chemistry; c) a means for aerodynamic control over chamber shape which may either be held constant for the duration of analyte delivery or modulated by feedback control during analyte delivery; and d) a means for active, dynamic feedback control over shape, duration, air flowrate, temporal envelope, and frequency of analyte sampling (sniffmg). Such feedback may be derived from examining the spatio-temporal response patterns from the sensor array produced by prior analyte sampling.

The sensing chamber can be optimized for its aerodynamic properties by placing the detectors in cavities of various shapes. In one embodiment, the sensors may be placed at a bend in the flow path. In an alternative embodiment, the sensors may be located on the side of the straight flow path.

The present invention provides the sensing elements that are composed of nucleic acid/fluorophore mixtures applied to removable sensor substrates. In one embodiment, thin films of nucleic acid/fluorophore mixture are deposited on a flat silk, plastic or glass substrate. In preferred embodiments, a nucleic acid/fluorophore mixture is deposited directly onto fibrous support made from silk, natural or synthetic cellulose, polymers, glasses, ceramics, metallic, or other materials using an ink jet printer. The use of fibrous dye substrates dramatically increases the magnitude of the response signals, which improves analyte detection and discrimination of the device. In an alternative embodiment, thin nucleic acid/fluorophore films can be suspended freely across a perforated removable solid support which is placed in the center of the air flow stream, thereby exposing both sides of the nucleic acid sensor to volatile compound analyte.

The sensing device according to the present invention uses interchangeable, removable sensors or sensor elements comprising a support wherein nucleic acid/fluorophore complexes are attached. Easily removable sensors facilitate rapidly changing sensing sites for improving the sensitivity and optimizing discrimination for specific analytes in a variety of sampling applications. This feature further provides for rapid screening of different nucleic acid/fluorophore mixtures for evaluating new nucleic acid sequences and or structures or different fluorophores for use in sensors and also for evaluating analytical detection algorithms.

The size, thickness and surface area of sensor element sites may be modified to optimize sensitivity and discrimination and to efficiently couple sensor elements to light sources and detectors. Generally, a larger sensor geometric area and a close matching of the sensor element geometric area with photodetector area will provide better sensitivity.

The cross-reactive sensor array of the present invention may comprise either narrow or broadly responsive sensor elements. The number of sensor array elements can be configured for specific sampling application requirements. Specific sensors for defined analytical tasks can be chosen from among the many possible sensing element sites present in the array. Sensor and array configurations may be modified through the addition of LED-sensor-photodiode-filter channels depending on the requirements of a particular analyte discrimination task.

In one preferred embodiment, multiple sensor arrays and array substrates may be deployed in the sampling chamber. Such multiple arrays may comprise a series of hierarchically organized sensor arrays such that the first interaction and sampling of the analyte is with a broadly responsive sensor array and, subsequently, the analyte sample is automatically diverted for additional sniffs, on the basis of analytical information fed back from the computer, to specific second order arrays designed to detect and identify the specific type of analyte. Thus, a plurality of sensing arrays may be arranged hierarchically so that ever finer discriminations can take place successively along the pathway. Additionally, the longevity of sensors can be extended by redundant arrays that are protected from exposure until needed, by delivery of analytes as short pulses, and by reducing light exposure by rapidly pulsing LEDs. To further reduce light exposure, low light excitation levels can be used if high sensitivity photodetectors such as avalanche photodiodes are employed. Rapid short pulsing of analytes prevents sensing surfaces from saturating, thereby improving sensor recovery following analyte exposure.

For enhanced, smart mode operation, the number of array sensors used in sampling or detecting an analyte may be modified, in real-time during either actual sampling or post-sampling data analysis using “on-the-fly” intelligent feedback control. By way of example, if a specific sensor is unresponsive to a particular. analyte sample, the corresponding sensing channel may be automatically removed from consideration by a smart sampling or analysis algorithm which provides feedback control to the microcontroller. In addition, the weighting of individual sensors in the analysis and detection algorithm may be adjusted based on the signal contribution of individual sensors. Given that individual sensors have different breadths and peaks of response, sensor weighting will vary for different analytes.

In one preferred embodiment a 16 or 32 channel sensor array is employed. For example, it is anticipated that an optimized array of thirty-two sensor elements should have the capability of detecting and discriminating at least 1000 different analyte types. Because the nucleic acid/fluorophore-based sensor materials employed provide almost infinite diversity in their variety and therefore their analyte detection capability and can be selected to have appropriately broad spectra of response, different optimized sensor arrays can be selected for particular analyte detection tasks.

Typically, epi-illuminating optics are employed in conventional fluorescence sensing systems. Epi-illuminating optics require relatively complex dichroic mirror arrangements for each channel where a different excitation and emission wavelength is used. Thus, in the epi-illumination format an excitation filter, a dichroic mirror, and an emission filter are required for each wavelength. The sensing system of the present invention employs a trans-illumination configuration where only excitation and emission filters are needed. Since the epi-illumination mode typically requires critical optical component alignment and is sensitive to vibration and movement, the trans-illumination mode of the present invention is advantageous for robust, compact, portable sensing devices for field sampling of ambient environments.

A schematic diagram of the optical detection system of the present invention is provided in the block diagram of FIG. 9. FIG. 1B provides a cross-sectional view of the sampling chamber that schematically shows the configuration and relative orientation of individual LED-photodiodes-optical filters-sensor sets within the sampling chamber housing. For simplicity, the cross-sectional view in FIG. 1B shows only two sensing channels, comprising two LED-photodiode-filter-sensor channel pairings. FIG. 1A shows a view of a sixteen sensor array configuration. It is important to note that the partial array configurations shown in FIGS. 1A and 1B are merely used to demonstrate, by way of example, the relative orientation and positioning of the sensors, filters, photodiodes and LEDs in the sampling chamber and are not intended to indicate any limitation in the size of sensor arrays that may be employed in the present invention. The actual sensing device of the present invention may employ larger or smaller arrays and any number of sensing channels with corresponding LED-photodiode-filter-sensor sets. For example, in one preferred embodiment, 32 LED-photodiode-optical filters-sensor channel sets are employed. The number of sensor array channels may be increased or decreased depending on specific sampling applications and analyte discrimination requirements.

An example of the configuration and relative orientation of LEDs, photodiodes, excitation filters and emission filters, sensors and sensor array substrate is shown schematically in FIG. 10. While an eight sensor-LED-photodiode-filter module is shown in FIG. 10 by way of example, larger and smaller modules and arrays may be constructed based on specific sampling and detection needs. For example, in one embodiment, a 32 element sensor array may be assembled from four modules aligned side-by-side with eight sensors in each module. As shown in FIG. 10, a plurality of LEDs are mounted on black plastic support by drilling two columns of four 3 mm holes in a 2×4 array configuration. The LEDs are press fit into the mounting holes and may be readily removed for replacement. A photodiode support with the same dimensions is used for mounting a plurality of eight photodiodes in a 2×4 array configuration. Both the LED and photodiode arrays are mounted in columns with pair row spacing of about 6 mm center to center and interpair spacings of 8 mm center to center. Column spacing for both the LED array and photodiode array is 15 mm center to center.

As shown in FIG. 10, 12.5 mm diameter excitation filters are mounted on an approximately 30 mm×30 mm×6 mm excitation filter support formed by drilling four 12.5 mm holes in a black plastic support plate to accommodate the filters in a 2×2 array configuration. Other filter assembly configurations, containing a larger or smaller filter array with larger or smaller filters may be employed in other embodiments. A similar emission filter support with the same dimensions as the excitation filter support is fabricated for mounting four emission filters. The emission filters and excitation filters are mounted to their respective supports with conventional set screws. The resulting excitation filter support assembly is attached directly to the front face of the LED support assembly and the emission filter support assembly is attached directly to the front face of the photodiode support assembly with conventional mounting screws.

A plurality of nucleic acid-based sensor elements are applied either directly to a transparent sensor array substrate, for example a glass coverslip, as coatings or droplets. Alternatively, where porous or fibrous sensing elements are employed, these may be attached, for example, taped, glued, or clamped, to a transparent sensor array substrate, or suspended over openings or perforations in an array support which may be either transparent or opaque. Removable, interchangeable sensor array substrates, or array support substrates, can be mounted flush with the front face of the emission filter support using a substrate support holder. The substrate support holder is formed by attaching, for example by gluing, a shaped, preferably U-shaped substrate support frame and a shaped substrate support facing to the front fact of the emission filter support. The sensor array substrates, or array support substrates, are, for example mounted in a slot or channel formed by the substrate support frame, support facing and front face of the filter support. The substrate support assembly provides for rapid removal and replacement of the interchangeable array substrates or array support substrates.

The sensor array may comprise either a single sensor array module, as shown in FIG. 10, or a plurality of sensor modules aligned edge-to-edge to form a multi-module array containing a large number of sensor elements. The bottom edge of both the LED-excitation filter module support assembly and the photodiode-emission filter-sensor module support assembly are secured to a chamber support plate with conventional mounting screws. In this configuration, the excitation filter side of the LED assembly faces the sensor array side of the photodiode assembly. The LED and photodiode modules, or plurality of modules, are preferably aligned parallel to one another with spacing between the two modules adjusted to optimize illumination of the sensor array elements by the LED array. In one preferred embodiment shown in FIG. 10, this spacing is approximately 5 mm. In one preferred embodiment, a 32 sensor array is formed by mounting four eight sensor modules to the chamber support plate. Other configurations using larger or smaller sensor modules and a fewer or greater number of modules may be employed to accommodate smaller or larger arrays by adjusting the size of the LED, photodiode, filter and sensor supports and chamber support plate and adjusting the spacing between opposing LED and photodiode modules to optimize illumination of sensor array elements by the LED array.

Commercially available, optical bandpass excitation filters for LED light sources and emission filters for photodiode detectors were obtained from Andover Corp. (Salem, N.H.) and Coherent Inc. (Santa Clara, Calif.). While these filters are available in ¼ to 1½ inch sizes, ½ inch filters were used in the preferred embodiment. By way of example, FIG. 10 shows schematically the relative orientation, configuration and spacing of excitation and emission filters for an embodiment which employs 32 sensors and sensing channels. For simplicity, FIG. 10 shows only one of four eight-sensor modules employed in a 32 channel sensor array. In this embodiment, with four sensor modules, 16 excitation filters are arranged in a 2×8 array with a center to center distance of 15 mm. With this embodiment, each emission filter covers a pair of two adjacent photodiodes having a 6 mm center to center spacing. In this particular embodiment, the 32 sensor elements in the array were aligned with the center of the LED-photodiode pair sight line. Other embodiments are envisioned where each sensor channel has its own individual excitation and emission filter or where more than two sensor channels share each excitation and emission filter. For example, for YO-PRO and Oligreen dyes, an excitation filter of 450 nm with a 40 nm bandwidth, and emission filters with 550 nm with a 70 nm bandwidth can be used (Coherent Inc., Santa Clara, Calif.). Dyes such as BOBO-3 and Cy3(tm) require longer wave lengths which one skilled in the art is capable of selecting.

Illumination of sensor elements with excitation light energy may be accomplished with any appropriate light source. Thus, filtered light emitting diodes (LEDs), solid-state lasers, or incandescent light sources of the appropriate wavelengths for the dye indicators being used may be employed. In a preferred embodiment, each LED light is passed through an excitation filter matched to a specific sensor element dye excitation wavelength. Where excitation filters are employed, broad-band (“white”) LEDs with appropriate wavelength filters may be used.

Unlike other sensors, by providing individually filtered sensing channels, the present invention enables simultaneous sampling at multiple excitation wavelengths and multiple emission wavelengths with different sensor elements. The. present invention uniquely provides for individual control over the amplitude, duration, and duty cycle of illumination for each sensing channel in the array. Control over noise is exerted by feedback. Control over response to ambient light and optimization of signal detection, including reduction of fluorescent dye bleaching, is accomplished by switching and modulating LED output and coordinate amplifier detection at various frequencies, ranging from kilohertz to megahertz. Control over ambient light interference may be achieved by phase locked LED flashing and photodiode detection.

In the present invention, nucleic acid-based sensor elements are illuminated directly by focused, light emitting diodes (LEDs) of the correct wavelength for each sensor dye material. Other advantages achieved from using LED excitation light sources are low power requirements, cooler operating temperatures, and high light output over small area. Additionally, by employing LED light sources for each sensor channel, each LED channel can be rapidly and independently switched electrically without use of a mechanical shutter. The LED channels can be individually modulated electrically at high rates by feedback from the microcontroller. In addition, the LED channels can be individually filtered for presenting different excitation wavelengths in parallel, thereby avoiding serially and mechanically switching filters during array measurements.

Examples of LEDs useful according to the present invention for the nucleic acid based sensors include, but are not limited to Hosfelt #25-365, Ultra Bright Blue LED, rated at about 466 nm. Other LEDs useful according to the present invention can be selected according to wavelengths appropriate for each and every fluorescent molecule that can be attached to the nucleic acids as shown in the Table above.

The LED's are turned on and off under computer control. Since these devices can respond at high speeds, up to megahertz frequencies, they are typically flashed at kilohertz frequencies in order to reduce bleaching. Such switching speeds cannot be achieved using mechanical shutters. The rapid switching capacities of LED's are utilized to flash them on and off in order to reduce sensor bleaching during data acquisition, thereby reducing total light exposure by shortened duty cycle during sample sniffs. LEDs are rapidly flickered so that light is only on during the time when data are being taken and then turned off between data points and between trials.

While a variety of photodetectors such as photomultiplier tubes (PMTs), charge-coupled display device (CCD) detectors, photovoltaic devices, phototransistors, and photodiodes may be used for detecting sensor response signals, in a preferred embodiment, filtered photodiode detectors are employed. In another preferred embodiment, highly sensitive avalanche photodiodes may be employed. Photodiode detectors have distinct advantages compared to conventional CCD camera detectors since they enable independent control and modulation of individual channel optical filtering, current/voltage conversion, signal amplification, and temporal filtering. Other specific advantages are low power consumption, relatively simple electronic circuitry, high sensitivity, configurability, multiple array formats (e.g. circular, square, or linear arrays), fast high frequency response at megahertz frequencies, low noise, wide dynamic range, and use with low frequency circuits.

In the nucleic acid-based sensing device of the present invention, an array of filtered photodiodes is employed where each filtered photodiode is either aligned with one filtered LED or, altematively, groups of filtered photodiodes may be illuminated by a single filtered LED. The individual photodiodes are each aligned with an individual sensor element site with an optical emission filter that is appropriate for the specific dye employed by the individual sensor. Different emission filters may be used for each photodiode or, alternatively, one emission filter may be shared by multiple photodiodes. Photodiode signal noise is controlled by feedback. Additionally, feedback control is exerted over the signal sampling duration and time course. Differential signal inputs may be employed with a separate control sensor and individual sampling sensors. In one preferred embodiment, highly sensitive avalanche photodiodes may be used to permit lower required LED intensity for sensor excitation thereby reducing sensor photobleaching.

In one embodiment commercially available EG&G VTP 1232 photodiodes (EG&G, Inc, Gaithersburg, Md.) and 12.5 mm emission filters (Andover Corp., Salem, N.H. and Coherent Inc., Santa Clara, Calif.) were used. In a preferred embodiment, large area photodiodes (Hamamatsu part no. S2387-66R) are used. Specific emission filters used in conjunction with the photodiode detectors are discussed above.

While sensors may share the same LED, photodiode and excitation/emission filters, in alternative embodiments, separate LED, photodiode, sensor, and excitation/emission filters may be employed for each of sensor element and sensing channel. In one embodiment, individual sensor elements and sensing channels may employ different sensing materials, different excitation wavelengths, and/or different emission wavelengths simultaneously. One skilled in the art may increase or decrease both the size of the sensor array and number of sensing channels, following the teachings disclosed herein.

In one embodiment, all LEDs are powered by a single constant voltage circuit. The changes in fluorescence as a result of the odor interacting with the sensing material is detected by a photodiode and current to voltage (I/V) converter originally designed by Warner Instruments (Hamden, Conn.) and now commercially available from Red Shirt Imaging Inc. (Fairfield, Conn.). There is one I/V converter and amplifier/filter for each detector channel. The unique feature of this converter/amplifier configuration is that when the LEDs are activated prior to sample delivery, the background fluorescence signal produced by the sensor elements may be offset by resetting the amplifiers to a baseline value so that a full range of high gain amplification may be used to observe small changes in the signals generated by analytes during sampling. In addition, the amplifier board has the option for software control to be exerted over the gain and the filter time constants for all the channels. Photodiode output is digitized using a 12 bit A/D converter. In a preferred embodiment, each LED is powered independently by its own constant current circuitry. The output current of each photodiode is converted to voltage and digitized to 20 bits using an integrating preamp/AD converter IC manufactured by Burr-Brown (DDC112). The DDC112's provide separate gain control for each sensor channel. Circuitry containing two programmable logic devices (PLD; Xilinx part no. XC95108-15PC84C) generates the high speed timing control signals for the 16 DDC112 chips.

Thus, in addition to being able to manipulate the onset and duration of the illumination and of the sniff as described above, the time constants and gain of the amplifiers can also be controlled in real time during data acquisition. These hardware features offer distinct advantages for optimizing the response of the sensing device for detection, discrimination and identification of analytes or odors of interest.

Generally, the nucleic acid/fluorophore-based sensing system of the present invention analyzes spatial-temporal patterns of data output from nucleic acid-based sensor arrays in order to characterize and identify the delivered sample or its analyte components. Useable information from the sensing array is obtained from the pattern of sensor response activity generated by all sensor elements over time and is evaluated using statistical measures such as information theory. Pattern recognition algorithms including template comparison, neural networks, principal components analysis, etc. may be implemented either in conventional digital CPUs, in neuronal network simulator chips, or in analogue neuronal network computers. Additionally, algorithms based on biologically based neuronal connections from the olfactory system and other neuronal circuits in the brain may be employed.

The analytical circuits of the present sensing device provide the requisite hardware support for the detection, discrimination and identification capability of the sensing system.

The present invention uses temporal control over stimulus presentation and the examination of the resulting changes in sensor output over time. Unlike other designs, with the present invention analyte presentation to the sensing sites is carried out by negative pressure ‘sniffing’, rather than by positive pressure pulsing which requires samples to be enclosed in confined containers. Additionally, the present invention uses sniffing parameters that can be electronically modulated by feedback from via computer control and flow rate, sniff duration, and temporal profile can be adjusted and modulated for specific sampling environments and target analytes to detect ambient odors drawn into the sensing chamber. Sampling modulations can be carried out in real time so that subsequent sniffs can be modified by the preceding ones. With the smart sampling mode capability of the present invention, a computer turns the sniff on and off and can modulate and control sniff parameters during sampling.

Target samples of known analytes (odors), either pure compounds or complex mixtures, are required for training the sensing device and identifying unknown analytes in sampled fluids.

For all training runs, initially a clean air test sniff is first taken by initiating the automated sampling sequence which provides for turning on the LEDs, taking digitized data from the photodiodes, measuring background fluorescence and storing this in memory, turning on the sniff pump, turning off the pump, terminating data acquisition, and turning off the LEDs. The device is then trained for target analytes by placing the target analyte sample container into position and initiating the automated sampling sequence. The sequence of sampling and data acquisition events for target analytes is the same as for the air baseline sample. This training sequence is repeated for each target analyte of interest and response data are stored in the microcontroller computer RAM memory module.

After analyte presentation and data acquisition using a device, such as a device described in the U.S. Pat. No. 6,649,416, evaluation circuits and algorithms characterize the spatio-temporal response data of the array either via pattern recognition algorithms, template matching, a neural network, statistical analysis, or other analytical methods known to be useful for data analysis from multiple points. Results may be displayed on screen, spoken by voice synthesis, or plotted as a three-dimensional response surface of fluorescence changes from each sensor at each time point during sampling. If sensing device is on robotic vehicle, results are processed for feedback control and decision is made to stay on course or execute an appropriate maneuver.

Optionally, where multiple samples or complex mixtures containing multiple analytes are being sampled, with data sampling and acquisition modifications based on intelligent feedback via smart algorithms. Thus, real-time, on-the-fly feedback can dynamically modulate either LED, photodiode, or sniffing hardware settings, or alternatively, analyte sampling parameters such as, sample duration, rise time, relaxation time, delay from previous sniff, amplifler gain and time constants may be modified. These modifications may be imposed on the next data acquisition within the same sampling trial until detection and identification of the analyte occurs.

The software program explicitly controls the pre-bleaching phase, the duration for which the LED's illuminate the sensors, the onset of data acquisition, the application of the analyte, the duration of analyte presentation, the cessation of analyte application, the duration of the integration time for each data point, the number of time points, and the interval between time points. All of these parameters can be modulated either by direct operator intervention or, alternatively, by programming the microprocessor with smart algorithms that modify the sampling, data acquisition, or analysis steps through real-time feedback control.

The data are filtered, smoothed, statistically evaluated, compared with libraries of stored templates for odor identification, and/or operated on by any of the algorithms discussed below. The data are typically stored in memory as an array of numbers representing the temporal changes in fluorescence in each sensing channel.

### 1. Detection Methods and Algorithms

A. Evaluation of Synchrony, Response Signals and Noise Characteristics

To improve the detection and discrimination capability of the sensor of the present invention, additional algorithms may be employed to evaluate “synchrony” of response data across different sensor elements to identify small response signals and reject noise. Evaluation of “synchrony” refers to analyzing how signals coming from identical sensors are similar in the context of when they occur during the sniff cycle. The field that encompasses analytical algorithms is very large and many analytical approaches are available. Due to the features of the present invention, such as the use of multiple detector channels with different wavelengths, use of single or multi-pulsed analyte presentation, and the ability to acquire data from sensor elements in parallel rather than serially, the design of the present invention enables consideration of a number of alternative algorithms beyond those that are conventionally used in artificial noses. Additionally, in preferred embodiments algorithms which are based on biological circuits may be employed (see J. White, et al., Biol. Cybern. 78:245-251(1998); J. White, et al., Anal. Chem. 68(13):2191-2202 (1996), which publications are incorporated herein by reference in their entirety). The device of the present invention may employ synchronously occurring signals in some embodiments since sensor response data are acquired simultaneously in parallel.

Detection Algorithms

The degree to which the response matrix of a test substance corresponds to one of the target analyte library matrices stored during the sensor training phase can be evaluated in a number of ways.

In one preferred embodiment, the rising phase of each sensor signal is fit by an exponential function containing two parameters describing the signal amplitude and rate of change. A matrix of these parameters is then used to represent the sensor array response. Matches are determined from the sum of the squared differences between each parameter in the test matrix and the training matrix. The smallest sum is used to identify the best target analyte match.

In an alternative preferred embodiment, a supervised, for example back propagation, neural network approach may be employed. Examples of these methods are provided in J. White, et al. “Rapid Analyte Recognition In A Device Based On Optical Sensors And The Olfactory System”, Anal. Chem. 68(13):2191-2202 (1996) and S. R. Johnson, et al., “Identification Of Multiple Analytes Using An Optical Sensor Array And Pattern Recognition Neural Networks”, Anal. Chem. 69(22):4641-4648(1997).

In another preferred embodiment, analytical circuits based on the olfactory system may be employed as disclosed by J. White, et al., “An Olfactory Neuronal Network For Vapor Recognition In An Artificial Nose”, Biol. Cybern. 78:245-251(1998).

In another preferred embodiment, unsupervised neural networks may be used. Principle component analysis and multidimensional scaling are, in effect, unsupervised statistical methods for reducing dimensionality. Generally, unsupervised neural networks organize high dimensional input data into lower dimensional representations. For example, assuming one embodiment of the present device with 32 sensors and 20 time points, a total of 640 data points may be collected. In this embodiment, each analyte presentation can thus be thought of as a point in 640-dimension space, which, while difficult to visualize, may be mathematically manipulated. By averaging across sensors and time, the data dimensionality may be reduced, but typically data dimensionality above about four dimensions is rather difficult to visualize.

Self-organizing maps (SOMs) are unsupervised neural networks that reduce data dimensionality. Such SOM methods are attractive for representing artificial olfactory system data because they give a visualization of “odor space”. In other words, a map of relationships among various analytes can be produced during training; then during testing, the location of a test analyte on the ‘map’ indicates the relationship of the analyte with respect to this ‘space’. Thus, SOMs may help to visualize relationships among analytes, rather than simply indicating the similarity of an unknown analyte to a target. Examples of SOM approaches which may be particularly useful for analyte detection, discrimination and identification are disclosed by T. Kohonen. et al., “SOM-PAK: The Self-Organizing Map Program Package”, Report A31, Helsinki University of Technology, Laboratory of Computer and Information Science, Espoo, Finland (1996) and T. Kohonen, Self-Organizing Maps, Series in Information Sciences, Vol. 30, 2nd ed., Springer-Verlag, Heidelberg (1997), which publications are incorporated herein by this reference.

Sampling and Detection Parameter Modulation

Upon evaluation of the response matrices generated by the standards used for training, modifications in sniffing parameters, gain settings, and/or filter settings may be made for actual sampling of ambient fluids. In a standard operating mode, these modifications may be made through interventions of an operator who manually changes sampling and data acquisition parameters through the programmable microcontroller or by keyboard entry. In alternative smart operating modes described in subsequent sections, these modifications may be made automatically, on-the-fly by smart sampling and detection algorithms that direct microcontroller operations.

Whether and how much such modification improve sensing performance may be evaluated by examining sensor responses after feedback and determining, by some pre-determined or analytically-derived criterion, whether current sample data are better or worse than data obtained on a previous run. Modifications may also consist of differentially weighting the influence of sensors, so that those sensors that give the best signals have a greater impact in the recognition algorithms. This can be done in a number of ways, such as eliminating sensors that give little or no signal so as to reduce noise, normalizing the remaining signals to some standard value in order to use the maximum range available, or changing the analyte sampling and stimulus acquisition paradigm to optimize sniff sampling parameters.

“Smart Mode” Operation

One example of an embodiment of the smart mode sampling capability of the present invention is where the number and duration of analyte samples taken during a sample session are controlled by way of real-time feedback and control loops for improving detection, discrimination and identification of analytes. In other embodiments, alternative smart mode parameters and device sampling configurations may be manually or automatically selected during training and sampling via device menu options. Smart mode sampling configurations may be used alone or in a variety of combinations and permutations. In one anticipated embodiment, an automated training algorithm may be employed to optimize parameter selection and sampling configuration in order to provide the best detection and discrimination capability for specific analytes of interest. Specific examples of alternative smart mode sampling options and parameter configurations are described below.

Sampling Parameters

Sniff Parameters—Sniff Duration

For sensors that respond slowly to a particular analyte, increasing the sniff duration leads to increased signal amplitude and hence improved detection accuracy.

Sniff Parameters—Number of Sniffs.

In the simplest implementation, signals across multiple sniffs may be averaged to improve signal-to-noise. However, different sensors exhibit different long-term responses to multiple sniffs (providing either increasing signal, decreasing signal, or constant signal over a series of sniffs). Monitoring these changes over sniffs (rather than simply averaging the signals) could provide additional information for analyte discrimination.

Sniff Parameters—Sniff Dynamics (Rise Time, Fall Time).

The rate and extent of sample chamber valves opening and closing may be controlled to modify sampling (sniff) dynamics.

Changing the sniff dynamics may enhance differences in the rising and falling phases of the sensor response.

Sniff Parameters—Sniff Velocity.

In one anticipated embodiment, a digital-to-analog line may be used to control a transistor that changes the voltage supplied to the sniff fan and thereby alter fan velocity. Changing sniff velocity, in conjunction with changes in sniff duration, can provide optimized exposure of the sensors to particular analytes.

Sniff Parameters—Exhalation Velocity.

As with changing sniff velocity, a change in exhalation velocity in an embodiment with two fans would alter the rate at which analyte is purged from the. sensors. In a system with a single fan, the velocity of that fan between sniffs can be similarly altered. The dynamic sensor response may then be monitored in subsequent sniffs for improved analyte discrimination. LED Intensity.

While higher LED intensity leads to more rapid photo-bleaching and sensor degradation, it also tends to yield larger sensor response signals during analyte exposure. In one smart mode embodiment, normal sampling would be made at lower LED intensity and, where small response signals are present, LED intensity may be increased incrementally until reliable response signals are produced for analyte detection. This smart mode would tend to extend sensor lifetime by operating at minimum LED intensity to reduce photobleaching.

LED Wavelength.

The excitation wavelength of the LED may be modulated. LEDs are commercially available that produce three separate wavelengths. The wavelength of conventional LEDs may be modulated by changing applied voltage and flicker frequency. The capability for changing LED wavelength may permit the device to optimally excite the sensors and to change that excitation over sniffs to improve discrimination.

Amplifier Gain Settings.

Under typical sampling conditions, the highest gain settings are employed. Under such a condition, some analytes produce sensor signals that saturate the amplifier. By providing for adjustment of gain settings during smart mode sampling, if an amplifier channel saturates, an additional sniff at a lower gain setting would provide more accurate time course and amplitude information.

Amplifier Temporal Filter Settings.

In embodiments incorporating amplifiers containing integral temporal filters, changing the filter settings may be used to improve the signal-to-noise characteristics of the individual sensor channels. Data acquisition and A/D conversion are closely correlated with LED pulse timing. However, some detection enhancement may be achieved by modifying the timing of data acquisition during an LED pulse for improved signal discrimination for specific analytes; modulation of this parameter may therefore improve detection and identification of certain analytes.

Gain and Temporal Filter Settings for Individual Channels.

While one current embodiment of the amplifier electronics allow manipulation of gain and filter settings globally (i.e. gain and filter changes apply to all channels simultaneously), in alternative sensor embodiments, individual sensor channels may also be manipulated for smart mode sampling and detection.

Smart mode training and sampling procedures using these and other parameter variations are discussed in greater detail below.

Smart Mode Training

Smart mode training can be divided into two sections: first, the parameters defining the “primary” sniff are determined, followed by a determination of parameters for any “secondary” sniff(s) that may be necessary. The constraints for the two sets of parameters are different: The primary sniffs are applied at regular intervals over long periods of time and should have minimum impact on sensor lifetime since they expose the sensors to as little light as possible to reduce photobleaching and to as little analyte as possible to prolong sensor lifetime and shorten recovery time. Secondary sniffs are intended to generate signals that produce better discrimination.

Photobleaching and Bleach Runs

Exposing a fluorescent sensor to prolonged excitation light produces photobleaching, decreasing the fluorescent output of the sensor. This fluorescence recovers over time after the excitation light is turned off. In embodiments where sensors are exposed to prolonged excitation light during acquisition of response data at variable intervals, there appears to be more variability in sensor response. Preferably, response data are acquired at regular 15 second intervals. Sensor bleach runs establish this regular interval before data are actually acquired. The bleach runs are repeated until the signals from the sensors stabilize. In preferred embodiments using short excitation light exposures (1-5 ms), variability across sniffs due to photobleaching is greatly reduced.

In embodiments using longer excitation light exposure, bleach runs are acquired either with or without sniffing a blank air sample. The response matrices from these runs are compared to the previous run by calculating the sum of squares (SS) difference for all data points. For the first run, the comparison is to a matrix of zeroes. If the SS difference is stable, where successive SS differences change little, training target sampling is initiated. If the SS difference is unstable, a 15 second inter-run delay time is used and then the bleach run is repeated. While the operator may evaluate the SS difference stability visually, this process may be automated by setting a criterion which provides for minimum changes in successive SS differences; when that criterion is reached, the program continues and training target sampling is initiated.

Smart Nose Testing

Smart Nose testing a single analyte can occur in two stages. First, a primary sniff is taken and, if the primary sniff produces a good match to a target, that match is reported. Secondly, if the primary sniff does not produce a good match, one or more secondary sniff(s), if defined by training, are taken. If a match criterion is not reached, the matching difficulty is noted and the closest match reported. If the quality criterion is reached, the match is reported.

The photodiodes useful according to the present invention are generally more sensitive than and have larger dynamic range than individual pixels of conventional CCD camera detectors. The detection surface area of individual sensor photodiodes in the present device is larger than individual pixel areas of conventional CCD camera detectors. Additionally, due to the surface area of the LEDs and photodiodes employed in the present invention, larger sensor element areas may be employed and sampling is conducted over a larger geometric surface area of individual the sensor elements. Furthermore, the high porosity high surface area sensor substrates of the present invention, further enhance sensor response signals due to a substantial increase in sensor surface area to volume ratios and the volumetric sampling of sensor response signals generated within a three-dimensional substrate-sensor volume.

The enhanced sensitivity of the present sensors may be further augmented by utilizing multiple layers of sensing material ‘suspended’ in the air stream, employing larger surface area sensor elements and larger surface area photodiodes, and/or using replicates of multiple identical detectors in the sensor array from which signals are combined electronically. Replicates of different sensing materials may be incorporated into different sensor channels. Using replicates provides advantages not only with respect to the duplication of data to verify measurement reproducibility, but also with regard to reducing non-correlated noise from electronic components such as amplifiers.

The invention further provides a method of selecting a nucleic acid capable of responding to a vapor phase analyte, said method comprising: a) contacting the nucleic acid labeled with a fluorophore with an analyte in vapor phase; and b) measuring the emission proflile of the fluorophore in the presence and absence of the target analyte, wherein a difference in the emission profile indicates that the nucleic acid is responsive to the analyte in vapor phase.

The nucleic acids according to the method can be prepared by any method known to one skilled in the art including, but not limited to oligonucleotide synthesis using method described earlier, polymerase chain reaction (PCR) using any DNA as a template, or isolating nucleic acids from any source, including but not limited to eukaryotic and prokarytotic cells, nucleic acid libraries in bacteria, cosmids, yeast artificial chromosomes and such.

Nucleic acids may be labeled using any fluorescent label and method known to one skilled in the art. In one embodiment, the nucleic acids are labeled with Cy3(tm) label. A set of nucleic acid oligomers are designed, wherein the internal sequence is a random sequence and the N- and C-terminal ends have an essentially same sequence or an anchor sequence. An example of a random oligo nucleotide with random 20-mer sequence in between is T(15)CCN(20) AAACATTGCGAAGAAA (SEQ ID NO: 6). Such random primers with fixed anchor ends can then be used to create a library by amplifying nucleic acids isolated from any source, such as bacterial DNA. Once the random sequences are amplified, they can be cloned into a library, for example a plasmid library, using methods known to one skilled in the art. Such libraries can then be amplified using, for example, PCR with a forward primer haveing a sequence T(15)CC in combination with a Cy3(tm) labeled reverse primer T*TTGTAACGCTTCTTT (SEQ ID NO: 7).

In accordance with the present invention, if the nucleic acid is internally labeled, any position is acceptable. For example, the label can be located in or near the 5′-end, or in or near the 3′ end. Additionally, applied dye such as YO-PRO and Oligreen can also be used.

Once the nucleic acid is labeled it is purified. Purification may be performed using any method known to one skilled in the art. In the example outlined above, oligo dT spin columns (available, for example from Amersham Biosciences Corp., Piscataway, N.J.).

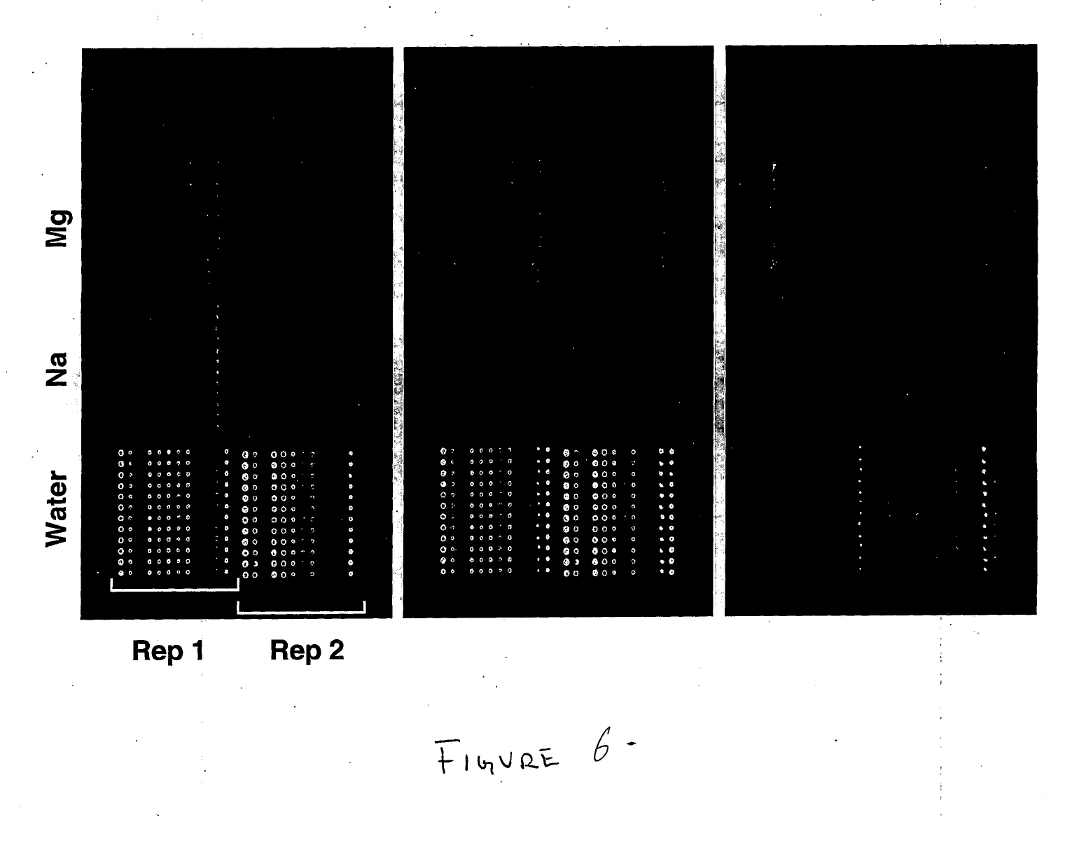

The microarray slides useful according to the present invention can be produced using a variety of surface substrates and methods of depositing nucleic acids on the surfaces. For example, glass coverslips containing spots containing thousands of different nucleic acid-fluorophore sequences, for example, Cy3-labeled DNA sequences, can be prepared using a robotic microarray spotter and let dry. The glass coverslips can then be put to a chamber, for example a chamber shown in the FIG. 7, and analyzed before exposure to a vapor phase analyte and during or after exposure to the vapor phase analyte.

The comparison of the before and during and/or after images can be done electronically by subtracting the before image from the during and or after image (for example, FIG. 6). Difference in the intensity of the fluorophore emission patterns in the images indicate that the nucleic acid is responsive to the vapor phase analyte. The difference in the emission pattern may be increase or decrease of the intensity of the emission between the before and during and/or after image. The decrease of at least about 2%, 5%, 10-15%, 20%, 30%, 40%, 50%, 60%, 70%, 80%, 90%, 95%, 98% or more and up to 100% is considered as indicative of a nucleic acid capable of responding to the odor. The increase in the during and/or after image of at least about 1%, 3%, 5%, 10%, 20%, 30%, 40%, 50%, or more including but not limited to 51-100%, 200%, 300%, and 1000%, compared to the before image is considered an increase indicating that the nucleic acid is responsive to the analyte in vapor phase.

The sensor responses may be varied by the use of salts or other chemicals present in the buffer of the nucleic acids, before they are deposited on the substrate, or different substrates or other during the preparation of the sensors.

Examples of salts include but are not limited to 50 mM MgCl₂, 50 mM SrCl₂, 50 mM CoCl₂, 50 mM CsCl, 50 mM ZnSO₄, 50 mM UO₂ (NO₃)₂, 50 mM CaCl₂, 50 mM BaCl, 50 mM CrK (SO₄)₂, 50 mM AlCl₃, 50 mM NaCl+10 mM Tris+50 μM MgCl₂, 50 mM NaCl+10 mM Tris+50 μM SrCl₂, 50 mM NaCl+10 mM Tris+50 μM CoCl₂, 50 mM NaCl+10 mM Tris+50 μM CsCl, 50 mM NaCl+10 mM Tris+50 μM ZnSO₄, 50 mM NaCl+10 mM Tris+50 μM UO₂ (NO₃)₂, 50 mM NaCl+10 mM Tris+50 μM CaCl₂, 50 mM NaCl+10 mM Tris+50 μM BaCl, 50 mM NaCl+10 mM Tris+50 μM CrK (SO₄)₂, 50 mM NaCl+10 mM Tris+50 μM AlCl₃.

Examples of cations useful according to the methods of the present invention in testing the optimal conditions for nucleic acid-fluorophores for their responses to vapor phase analytes include, but are not limited to: Ag-based on papers that suggest silver increases Cy3(tm) fluorescence in microarray; Re-based on paper that Rhenium causes superconducting-like resistance in DNA; transition metals, also want to test different oxidation states of the transition metals (Cr, Co, and Zn already tested); Alkali metals, LI, Rb, and Fr (Na, K, and Cs already tested); Alkaline Earth Metals, Be (Mg, Ca, Sr, Ba already tested); Lanthanide and Actinide Series, use those which are not poisonous or radioactive, (U0₂ already used); Groups 3a-6a: use those which have ionic forms soluble in water (Al already used)

The following are anions useful according to the present invention in testing the nucleic acids for their responses to analytes in vapor phase: Cl, NO₃, and SO₄.

Substrates as listed elsewhere in the application, such as different plastics and surface modified substrates, such as silanized substrates, can modify the response of the nucleic acid to a vapor phase analyte and should be taken into consideration when testing the nucleic acids and later when constructing the actual sensing array useful in detecting vapor phase analytes according to the present invention.

### EXAMPLE 1

The portable EVID and a schematic overview of the EVID's sensing chamber, sensors, optical components, sniff mechanism, and computer control lines are shown in FIG. 1. The EVID uses an array of sensors that change their fluorescence intensity upon exposure to brief pulses of airborne analytes (e.g., “odorants”). The EVID in its present form contains 16 sensors that can be illuminated and observed at 16 different excitation and emission wavelengths. The'sensors are placed along a narrow chamber through which ambient air is drawn (see below). The optical elements for illuminating and monitoring the sensors are positioned along the sides of the chamber (FIG. 1B). Excitation light is produced by LEDs providing wavelengths appropriate for the sensors being used (e.g., 460 nm and 530 nm).

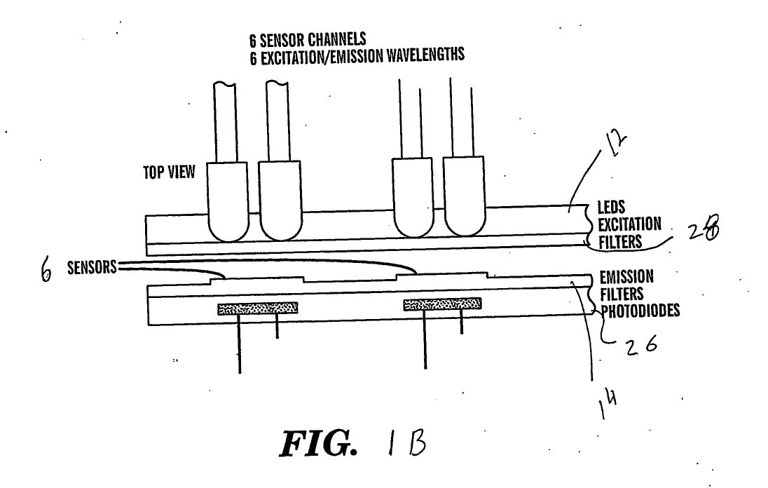

Dye-labeled DNA can act as an analyte sensor. As an initial test of whether DNA stained with a fluorescent dye responds to analytes, sensors were constructed from a standard 2.9 kb pBlueScriptSK plasmid mixed with YO-PRO dye (Molecular Probes, Inc.) and dried onto a substrate material (see method details below). Sensors made from YO-PRO alone and rinsed for 5 min in 70% ethanol showed no analyte responses. (FIG. 2A). A sensor made by mixing a small quantity of plasmid with YO-PRO, however, produced a large and rapid decrease in fluorescence upon exposure to propionic acid, and smaller changes to water, methanol, and triethylamine (FIG. 2B).

Tests with double-stranded DNA showed no sequence effects. To begin testing whether different sequences of double-stranded DNA per se can produce sensors of different analyte response profiles, two oligonucleotide oligomers were synthesized that were composed of solely GC or AT and were designed to form hairpin structures. Although differing significantly in primary sequence, the two sensors made from these hairpins had similar analyte response profiles. The hairpin sensor responses were also qualitatively similar to the sensors made from pBluescriptSK DNA.

Analyte dilutions as fractions of saturated vapor were: Water, 10⁻¹; methanol (MeOH), 10⁻¹; triethylamine, 10⁻²; and propionic acid, 10⁻¹. Each trace represents the mean of 10 presentations; error bars indicate +/−1 S.D. For experiments with DNA-based analyte sensors, similar methods were used for each type of sensor. Briefly, DNA in solution was diluted to the desired concentration (0.2-40 ng/μl) in TE (10 mM Tris, 0.5 mM EDTA). 20 μl of dilute DNA was mixed with 1 μl concentrated dye stock and incubated at room temperature for 5 minutes. Dye-only controls were made of 1 μl dye stock in 20 μl TE. Sensors were made on a substrate of acid-washed 16xx silkscreen (10 mm×12 mm). DNA/dye mixtures were pipetted onto the substrate and allowed to dry for 25 minutes. Each sensor was rinsed in 70% ethanol for 5 minutes, allowed to dry, then attached to supports on glass coverslips for testing in the EVID.

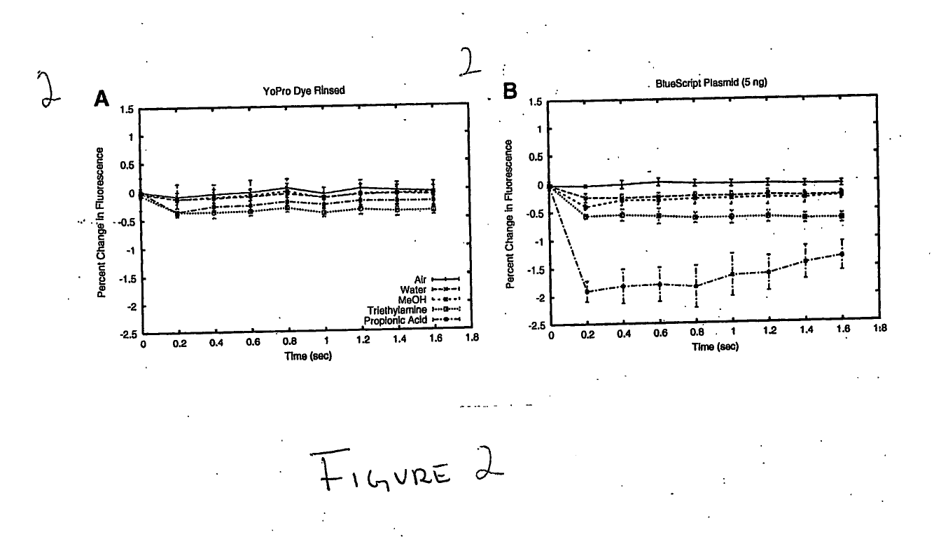

Single-stranded DNA sequences can show differential analyte responses. As a further test of whether differences in DNA sequence can produce sensors with different response profiles, sensors made from single-stranded DNA stained with the fluorescent dye OliGreen (Molecular Probes, Inc.) were tested. A sensor made from the OliGreen dye alone showed a decrease in fluorescence upon exposure to propionic acid, but little change with other analytes (not shown). This response was not eliminated with longer rinse times of 10 and 15 min. Sensors made from Oligo dT and oligomer DS003 showed enhanced signals to propionic acid and the other analytes tested (DS003 shown in FIG. 3A). The response profiles of these two sensors were similar to each other, and were also similar to the responses of the double-stranded DNA sensors made with YO-PRO (FIG. 2B).

A sensor made with the AJOOI primer sequence, however, had a markedly different analyte response profile (FIG. 3B). This sensor showed an increase in fluorescence in response to propionic acid and methanol, with relatively little change to the other analytes tested. While other DNA-based sensors showed responses to propionic acid, none showed as strong a methanol signal as this AJ001 sensor.

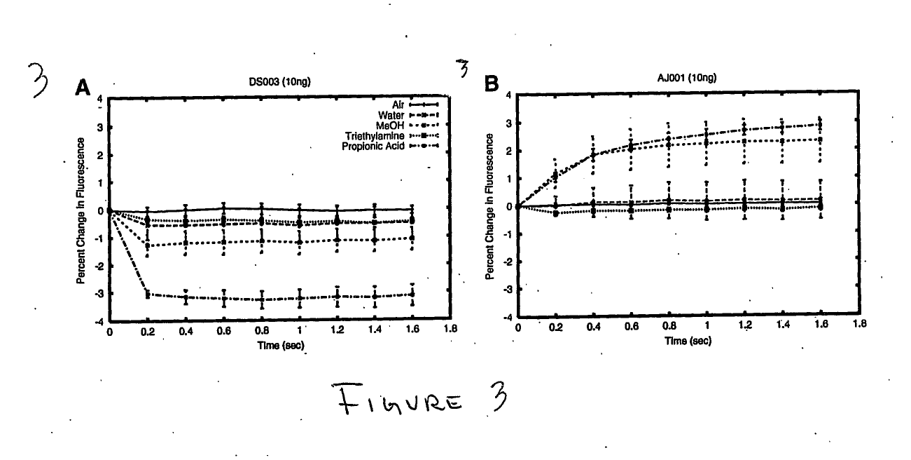

With applied dyes such as OliGreen, there is little control over how the dye interacts with the DNA sequence. In order to define the dye-nucleotide interaction explicitly, we tested oligonucleotides with the fluorescent dye Cy3(tm) covalently attached to the 5 ′ end during synthesis. Sensors made from Cy3(tm)-labeled sequenccs can show distinctly different analyte response profiles. The LAPP1 sensor (FIG. 4A) showed good sensitivity to propionic acid and triethylamine (detection limits at dilutions of about 10⁻³), and less sensitivity to methanol, DNT and DMMP (detection limits at dilutions of about 2×10⁻²). In contrast, the LAPP2 sensor (FIG. 4B) showed good sensitivity to triethylamine (detection limit at dilutions of about 10⁻³), less sensitivity to DMMP (detection limit at dilutions of about 2×10⁻²), and no response to propionic acid, methanol, or DNT, even at high concentration (10⁻¹ dilution).

It is important to emphasize that certain DNA-based sensors, such as LAPP1 shown here, respond to DNT. Besides the nitroaromatic sensing polymer developed by Dr. Timothy Swager (MIT), no other fluorescent sensor types that we have tested respond to DNT. Additionally, LAPP1 responds to DNT at dilutions down to 2×10⁻², or approximately 6 ppb, indicating that these sensors are capable of detecting low vapor-phase concentrations of some analytes.

### EXAMPLE2

(SEQ ID NO: 1)

| | |
|---|---|
| LAPP1: | 5′ GAG TCT GTG GAG GAG GTA GTC 3′ |

(SEQ ID NO: 2)

| | |
|---|---|
| LAPP2: | 5′ CTT CTG TCT TGA TGT TTG TCA ACC 3′ |

(SEQ ID NO: 3)

| | |
|---|---|
| LAPPAS: | 5′ TTT GGC TTT CTG GAA ATG GGC 3′ |

(SEQ ID NO: 4)

| | |
|---|---|
| LAJ001: | 5′ ACC AGG ACC TGA CTA AGC AGA T 3′ |

Oligomers LAPP1, LAPP2, LAPPAS, and LAJ001 were synthesized and labeled at the 5′ end with the fluorescent dye Cy3(tm) during synthesis (using Cy3(tm) phosphoramidite from Glen Research). The oligomers were stored in Tris-NaCl (10 mM Tris, 50 mM NaCl, pH 8) at 225 ng/ul, then diluted to a concentration of 50 ng/ul in distilled waterjust before use. Sensors were constructed by applying 20 ul of dilute oligomer solution to 10 mm×12 mm pieces of acid-washed 16xx silkscreen. Sensors were allowed to dry for at least 30 min at room temperature, then attached to supports for testing.

All sensors were mounted in the device and tested simultaneously. All were illuminated with excitation light at 540 nm (30nm bandwidth). Sensors made with LAPP1, LAPPAS, and LAJ001 were observed at 600 nm (10 nm bandwidth) and LAPP2 was observed at 610 nm (10 nm bandwidth). Vapors from propionic acid, triethylamine, methanol, DNT, and DMMP (dimethyl methylphosphonate, an organophosphate compound that is a simulant for Sarin) were presented to the device using an air dilution olfactometer at the indicated dilutions. For the graphs in the figure, each point in each curve represents the mean sensor response to ten 2 sec sniffs taken at 30 sec intervals; error bars indicate +/− one standard deviation. Signal amplitudes for the odorants are represented as multiples of the signal amplitude of background air (indicated by horizontal dashed line).

In the initial tests, the oligomer sequences LAPP1 and LAPP2 showed a distinctly different response profiles to this small test set of odorants. The LAPP1 sensor showed good sensitivity to propionic acid and triethylamine (detection limits at dilutions of about 0.001), and less sensitivity to methanol, DNT and DMMP (detection limits at dilutions of about 0.02). In contrast, the LAPP2 sensor showed good sensitivity to triethylamine (detection limit at dilutions of about 0.001), less sensitivity to DMMP (detection limit at dilutions of about 0.02), and almost no response to propionic acid, methanol, or DNT, even at high concentration (0.1 dilution). Sensors made with LAPPAS and LAJ001 sequences showed responses similar to LAPP2, but with smaller amplitudes. These data show that sensors that differ only in nucleotide sequence can exhibit different odorant response profiles. LAPP1 responds to DNT at dilutions down to 0.02, or approximately 6 ppb, indicating that these sensors are capable of detecting low vapor-phase concentrations.

### EXAMPLE 3

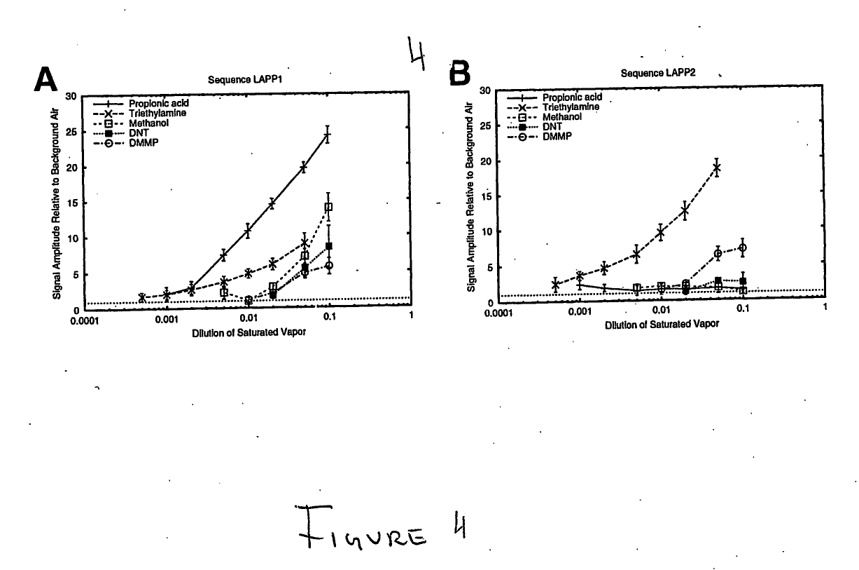

The dye-labeled DNA-based sensors described above can be selected using the system described herein. The strategy for finding different DNA sequences that respond to different analytes takes advantage of modern high-throughput methods and equipment for examining large numbers of DNA interactions rapidly. An overview of the approach is shown in FIG. 5 and is detailed in the following sections.

Prior to a large-scale sensor screen, details of the steps shown in FIG. 5 are established through a series of pilot experiments. The appropriate sequence length is determined, the actual sensor template is designed, and the necessary amplification and labeling conditions are established for generating large numbers of random DNA sequences for use as sensors using the methods described elsewhere in the specification. The amount by which the full sequence library needs to be diluted for effective screening is also be determined by testing different dilutions.

Determine sensor length. The single-stranded sensors investigated in our preliminary studies ranged from 18 to 23 bases in length. The minimum sensor length necessary for differential analyte responses, however, is unknown.

The sensor screen will be most effective if the final sensor library represents a significant portion of the original sequence library. The number of different sequences in the original. library goes up as 4^{no.bases}, so a sequence length of 20 bases would yield an original library of approx. 10¹² sequences, far greater than can be screened.

To determine a minimum effective sensor sequence length, LAPP1 and LAPP2 sequences labeled with Cy3(tm) are used as described. Sections of the two sequences are swapped, beginning with a swap point at the mid-point of the two sequences. A change in the analyte profile of either original sequence indicates that an effective sensor sequence is longer than the swap point.

The swap point is moved closer or farther away from the 5′ end (where the dye is attached) as necessary until there is no change in the analyte response profile. That point defines the minimum sensor length.

Design sensor template. Once the minimum sensor length is determined, the sensor template is designed. At least two possible amplification strategies are used: polymerase chain reaction (PCR) and primer extension (step 4 in FIG. 5). For PCR amplification, the template will consist of a random sensor sequence (length determined above) flanked by two anchor sequences. Alternatively, primer extension can be used for amplification, where the template will consist of a random sensor portion followed by a single anchor portion (see top of FIG. 5 for schematic representations of templates). In each of these templates, the anchor portion is complementary to the primer sequence(s) to be used for the amplification (one primer for primer extension, two primers for PCR). Each anchor/primer pair will be short in order to have as little effect as possible on the sensor responses, yet must be long enough to have a sufficiently high melting temperature for the amplification process An anchor/primer sequence of 13 bases will be investigated initially, which has an estimated melting temperature of 47° C.

Tests are conducted on both template types to determine the effects of flanking primer portions on analyte responses. Sequences containing known sensor regions (such as LAPP1 and LAPP2, shown in FIG. 4) flanked by one or two primer sequences will be synthesized along with attched dye label. The analyte responses of these sensors will be tested using the methods described elsewhere. Comparison of the analyte responses will determine whether PCR or primer extension will be used in the amplification (step 4) and hence will determine final template design.

Determine labeling procedure. Because any post-amplification procedures for attaching dye to the sensor sequences must be repeated about 10,000 times, the dye to the primer sequence is preferably, but not necessarily, attached so that it will be incorporated during amplification. In order to place the dye molecule as close as possible to the sensor portion of the sequence, the primer is labeled at the 3′ end by incorporating an amino-allyl modified dC or dT (Glen Research) during synthesis. N-hydroxysuccinimide functionalized Cy3(tm) (Amersham Biosciences) attaches the dye to the amino-allyl linker. After the dye reaction, a gel filtration purification step removes the unincorporated dye. The dye-labeled primer is then ready to use in the amplification step 4.

Determine amplification conditions. As mentioned above, PCR or primer extension is used to amplify samples of the sequence library (step 4 of FIG. 5), depending on the outcome of the analyte tests described above.

Optimal buffer conditions, reagent concentrations, and thermal cycling conditions are determined through tests with sensor templates containing a known sensor sequence and using the dye labeled primers described above. Amplification is monitored by spotting the amplified sensor sequence onto glass slides and testing for analyte responses using a microarray scanner.

Determine optimal dilution. The robotic spotter used to spot the senor library onto a microscope slide can apply approximately 10,000 sensor spots. In order to screen the largest possible number of sensor sequences, it is desirable for each spot to contain multiple different sensor sequences. Too many non-responsive sensor sequences in a spot, however, may obscure the signals from a single responsive sensor.

To estimate the number of non-responsive sequences that could obscure a responsive sequence, tests are conducted with the known sensor sequences LAPP1 and LAPP2. As shown in FIG. 4, LAPP1 responds to propionic acid whereas LAPP2 does not—propionic acid thus discriminates these two sensor sequences. Spots with varying integer ratios of dye-labeled LAPP1 and LAPP2 will be applied to microscope slides and exposed to propionic acid under the conditions that is used to screen the entire sensor library. The lowest LAPP1:LAPP2 ratio that still shows a propionic acid signal discriminable from LAPP2 alone provides an estimate of the number of sensor sequences per spot. This number of sensors per spot will then be set by the dilution in step 2 and the sample volume used in step 3 (FIG. 5).

Create a large-scale random library of different oligonucleotide-based sensors. The steps outlined in FIG. 5 and detailed above are followed to generate and amplify a sensor library, which is spotted onto microscope coverslips for screening with analytes.

A random sequence library is synthesized using the sensor template described above (step 1 of FIG. 5). The sequence library is diluted so that 1 μl samples contain at least one, and possibly multiple, different sequences (step 2). The 1 μl samples are put into 96-well plates (step 3).

Primer, bases, polymerase enzyme, and buffer are then added to each well. Four plates at a time (384 samples) are amplified using a PCT-225 PCR Tetrad thermal cycler (step 4). It is estimated that four plates can be amplified in a day, so amplification of the full 10,000 sample sensor library will require 26 days. A BioRobotics MicroGrid II microarray spotter is then used to produce sensor slides containing 10,000 spots of the amplified sequences (step 5). Multiple replicates of the spotted slides are generated, most of which are stored for future screenings.

The sensor library generated using the methods described above are screened with a set of explosive-related compounds and CWA simulants. Sensor spots showing responses to these compounds are further diluted, amplified, and spotted onto slides for additional testing with the analytes to locate the unique sensor sequences. The sequences are then determined using standard DNA sequencing methods.

A Packard BioChip Technologies ScanArray 4000 array scanner is used to scan a sensor slide while exposing it to analytes of interest. This requires the construction of a sealed slide holder so that analytes can be applied to the spots during scanning. One exemplary design is shown on FIG. 7. Examples of the analytes that can be used in the screening are shown in Tables 3-5.

Spots showing a change in fluorescence with analyte exposure are examined further. The amplified samples that produced the spots are further diluted to produce sub-samples containing individual sequences. These sub-samples are amplified again using the same amplification protocols developed above and spotted onto slides. The spots on these slides therefore contain individual sensor sequences. These slides are tested with analytes in the array scanner to identify individual sensor sequences for the odors of interest. The sequences in the sub-samples that produced the responsive sensors are determined using standard DNA sequencing methods. The final appropriate sequences are then synthesized, labeled, and tested directly in the EVID.

For each of the sensors identified in the sensor library screen described above, concentration-response functions are determined for each of the explosives, related compounds, and chemical agents listed in Table 3, 4 and 5. Using these data for individual sensors, an optimized sensor array is constructed and the lower detection limit for each of the compounds determined. These detection limits are compared to the reported sensitivities of commercially available devices, to toxicological data for the chemical agents, and, where data are available, to the behavioral thresholds of trained dogs.

TABLE 3
Example Explosives

| Target | Related compounds | Function | Reference |
|---|---|---|---|
| C-4 | Dimethyl | Taggant | |
| | dinitrobutane | | |
| | Dioctyl Sebacate | Plasticizer | |
| | 2-ethyl-1-hexanol | Solvent | |
| | Toluene | Solvent | Ron Ray, CDRI, |
| | | | pers. comm. |
| RDX | Cyclohexanone | Solvent | Jenkins and |
| | | | O'Reilly (1974) |
| TNT | DNT | Synthetic precursor | |
| | Dinitrobenzene | Contaminant | |
| | (DNB) | | |

TABLE 4
Example Chemical Warfare Agents/Blister Agents

| Target | Related compounds | Function | Reference |
|---|---|---|---|
| Mustard (H) | Dibutyl sulfide | Simulant | Pal et al. (1993) |
| | 2-Chloroethyl | Simulant | Jaeger et al. (1999) |
| | phenyl sulfide | | |

TABLE 5
Example Chemical Warfare Agents/Nerve Agents

| Target | Related compounds | Function | Reference |
|---|---|---|---|
| Sarin (GB) | Diisopropyl | Simulant | Pal et al. (1993) |
| | methylphosphonate | | |
| | (DIMP) | | |
| | Dimethyl | Synthetic | |
| | methylphosphonate | precursor | |
| | (DMMP) | | |
| | Isopropyl | Degradation | * |
| | methylphosphonate | product | |
| | (IMPA) | | |
| | Triethyl phosphite | Simulant | |
| Soman (GD) | Triethyl phosphate | Simulant | * |
| VX | Tributyl phosphate | Simulant | * |
| | DMMP | Simulant | Pal et al. (1993) |

* From http://www.dean.usma.edu/chem/Faculty/fountain/Fountainprcrsrch.htm

An air-dilution olfactometer, based on standard olfactometry concepts and modeled after a system used in dog studies (Hartell et al., 1998), is used to deliver controlled dilutions 6f analytes to the EVID. In the present configuration, filtered compressed air is fed to a bank of mass-flow controllers (Teledyne Hastings Instruments) to set flow rates through four air or analyte channels. Eight channels can also be used. One channel sets the background (diluent) air flow from 1 L/min to 10 L/min. The other three channels feed the air (flow rates from 10 ml/min to 10 L/min) through gas-washing bottles and other custom glassware containing analyte samples. Downstream of the analyte vessels, the analyte stream in each channel is controlled by electric valves (KIP Inc.), directing flow to exhaust or to a manifold bringing all channels back together. The manifold is connect to a glass analyte port, into which the snout of the EVID is placed for sampling. Analyte dilutions are determined by the relative flow rates through the diluent air channel and the analyte channel. Total flow rate is typically 10 L/min.

Sensors are characterized by their responses to the analytes over a range of concentrations. To collect concentration-response data for each analyte, a concentration series is delivered to the EVID using the olfactometer described above. An ascending series of concentrations are presented and ten sniffs at each concentration taken, with 30 sec between sniffs. Each concentration series starts with a dilution of 5×10⁻⁴ of saturated vapor concentration and ascend, in steps that approximately double the concentration, to a dilution of 10⁻¹ (i.e., 5×10⁻⁴, 10⁻³, 2×10⁻³, 5×10⁻³, 10⁻², 2×10⁻², 5×10⁻³, 10⁻¹). Data from the device is logged to a separate computer for analysis and display.

The detection limit for each sensor is determined by comparing the amplitude of the signal to that of clean air. The lowest concentration of an analyte that elicits a signal that is significantly different from the clean air signal is the detection limit of the sensor for that simulant. Detection limits for all the sensors available for all the analytes are determined in this way.

An optimized array of sensors, each with a low detection limit for one or more analytes, is selected. The detection limits of this array for each compound are determined using random presentations of the analytes at the dilutions listed above. Performance of the array is evaluated using signal detection theory and ROC curves. To evaluate the detection limits of the EVID determined in these studies, the values are compared to CWA toxicological data and to the sensitivities of other detection devices, including trained dogs.

### EXAMPLE 4

Preparation and testing of nucleic acids for their responsiveness to analytes in vapor phase.

DNA-Cy3(tm) Sensor Library. A set of DNA oligomers with random internal sequence and fixed ends (see FIG. 11) have been prepared by the Tufts University DNA/Protein Core Facility. Random nucleotide incorporation was used to generate the random portion of the oligomer. This random set provides DNA templates for amplification and labeling to produce a DNA-Cy3(tm) library for screening as described below.

Two methods are possible for initial amplification of the sensor library:

PCR. Polymerase chain reaction can be used to initially amplify the library sequences. The random oligomer library with determined primer and anchor sequences can be serially diluted, with the last dilution being no greater than 1:10, until it is calculated that each well in a microtitre plate contains, on average, one molecule of DNA. The DNA molecule(s) in each well is then be amplified and labeled by PCR through the reaction described below.

Bacteria A preliminary library has been constructed by putting the random sequences of DNA (FIG. 11) into plasmids using one of the commercially available single copy cloning kits (TOPO TA or Zero Blunt TOPO PCR) from Invitrogen. Plasmids were transformed into E. coli and colonies selected for expression of antibiotic resistance genes carried on the plasmid. The selection of bacteria with a drug resistant single copy plasmid allows one and only one DNA sensor sequence to be expressed in each colony. Colonies can be picked and grown in 96-well plates. After growth, a small portion of the bacteria can be lysed and the sensor sequence amplified and Cy3(tm) labeled in the wells using the primers as described below.

DNA Labeling Procedure. DNA can be fluorescently labeled using a variety of mechanisms and dyes. We have chosen Cy3(tm) to facilitate the use of existing microarray technology in our experiments. Importantly, our preliminary studies show that 5′ labeled single-stranded DNA-Cy3(tm) sensors respond robustly to odors. One method of 5′ Cy3(tm) labeling of DNA uses a 5′ capping reaction involving phosphoramidite chemistry during oligomer synthesis (reagents available from Glen Research).

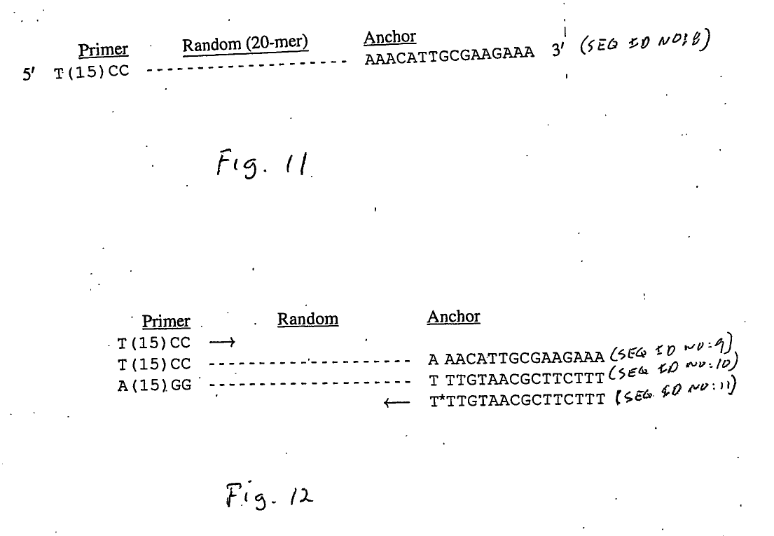

Although phosphoramidite chemistry provides a convenient method for attaching Cy3(tm) to DNA, it can only be used in synthesis reactions. This does not allow for attaching Cy3(tm) during amplification of the DNA sensor sequences. We have instead chosen to use a Cy3(tm) labeled primer in the PCR reaction to allow labeling at a defined location (FIG. 12). Cy3(tm)-labeled primer produced by first synthesizing an oligomer with a modified thymidine, Amino-modifier C2dT (Glen Research), at the 3′ end. C2dT was selected as an attachment site for Cy3(tm) because its short 2-carbon linking group gives the greatest possibility for electrochemical interactions between the DNA and Cy3(tm) attached to the two-carbon linker. Cy3(tm)-NHS ester (Amersham) was attached via an amide bond to the C2dT, using the NHS ester as a leaving group in the reaction (Amersham protocols were followed for this reaction). The primer was 3′ labeled so that the final DNA-Cy3(tm) product is 5′ labeled after removing all but the labeled base of the primer sequence using the restriction enzyme BsrD1. Results using Cy3(tm) linked to defined oligomers via a 5′ C2dT show the effectiveness of this method in creating DNA-Cy3(tm) odor sensors.

In other PCR studies, Taq polymerase has been used to add Cy3(tm)-linked nucleotides to a growing strand of DNA without interrupting the growth of the strand. Our method is similar, except that the label is located in the primer at the beginning of the growing strand instead of interspersed as Taq randomly adds labeled nucleotides. To remove unlabeled primer, which could exist if the reaction to attach Cy3(tm) to the primer is incomplete, Cy3(tm)-labeled primer was purified by reverse. phase high-pressure liquid chromatography (RP-HPLC). Preliminary results using this purification technique were successful and will be optimized.

Using the computer program Oligo no likely primer secondary structures or dimers were predicted, but PCR conditions need to be optimized for maximal amplification. Specific factors to optimize are MgCl₂ concentration, annealing temperature, and primer concentration. Optimization of the PCR is carried out with known sequences prior to preparation of the random library, but preliminary data has shown that a Cy3(tm) labeled PCR product of the appropriate size can be produced using our labeled primer. PCR products will be analyzed using 19% polyacrylamide gel electrophoresis. DNA bands will be examined using SBYR to show single-stranded DNA bands, and the Cy3(tm) fluorescent tag itself to show labeled bands.

The anchor sequence is removed by using a restriction enzyme, BsrD1 (New England Biolabs) to cut the anchor sequence from the label.

Although there are sticky ends remaining after BsrD1 digestion, the melting of the complementary strands in the purification process is more than adequate to separate the sticky ends. There is theoretically some loss in library yield due to random sequences which contain either the BsD1 restriction site or the primer sequence. Loss due to a BsrD1 restriction site in the random sequence is calculated to be 13×4¹² sequences. Loss due to random matching of the primer sequence is calculated to be 45 exact matches for each primer in each orientation or 46 total sequences lost. These losses combined represent less than 0.02% of the total library.

Purification of PCR Product. For method optimization, oligo dT spin columns (Amersham) are used to purify DNA. Polyacrylimide gel electrophoresis is used to analyze products. However, for a large DNA library, these methods are too slow and labor intensive. Separating labeled single-stranded DNA (ssDNA) from the complementary strand, primers, unused dNTPs, and reaction buffers is accomplished through washing after the desired ssDNA is bound to a substrate. Two possible methods are as follows:

Micro-titre plates with solid phase oligo dT in the wells are availabe from Sequitur, Inc. Hot DNA from the PCR reaction is placed in the wells and as cooling occurs, the poly(A) tail of the labeled DNA anneals with the oligo dT in the well. The rest of the PCR reaction mixture is then washed away.

Using TdT, a biotin conjugated base is added to the 3′ end of the labeled sequence. This is then bound to a strepavidin coated slide and the remaining material washed away.

Microarray Slide Production. Coverslips containing spots of thousands of different DNA-Cy3(tm) sequences are created using a robotic microarray spotter. Coverslips are then placed in a specially constructed chamber (FIG. 7).

Preliminary tests have been conducted using a prototype version of this chamber. Spots of 12 different sequences labeled with Cy3(tm) during synthesis were spotted onto a coverslip using a spotting robot (BioRobotics MicroGrid II), and the coverslip placed in the prototype chamber. Odors were delivered to the chamber in a controlled manner using a syringe pump. The coverslip was imaged using a microarray scanner (ScanArray 4000) before and after odor delivery.

Additional Results. The results outlined here indicate possible ways of modifying sensor responses by altering the salt content of the DNA-Cy3(tm) buffer during sensor construction and by using different substrates for making the sensors.

Effects of Different Salts. We have found that the salt content of the DNA-Cy3(tm) solution used to make the sensor can have an effect on sensor responses, both in terms of amplitude and in odor response profile.

The following salts were tested: 50 mM MgCl₂, 50 mM SrCl₂, 50 mM CoCl₂, 50 mM CsCl, 50 mM ZnSO₄, 50 mM UO₂ (NO₃ )₂, 50 mM CaCl₂, 50 mM BaCl, 50 mM CrK (SO₄)₂, 50 mM AlCl₃, 50 mM NaCl+10 mM Tris+50′ M MgCl₂, 50 mM NaCl+10 mM Tris+50 μM SrCl₂, mM NaCl+10 mM Tris+50 μM CoCl₂, 50 mM NaCl+10 mM Tris+50 μCsCl, 50 mM NaCl+10 mM Tris+50 μM ZnSO₄, 50 mM NaCl+10 mM Tris+50 μM UO₂ (NO₃ )₂, 50 mM NaCl+10 mM Tris+50 μM CaCl₂, 50 mM NaCl+10 mM Tris+50 μM BaCl, 50 mM NaCl+10 mM Tris+50 μM CrK (SO₄)₂, 50 mM NaCl+10 mM Tris+50 μM AlCl₃. In addition to these salt combinations, DNA-Cy3(tm) solutions tested contained 500 μM sodium borate buffer.

In these experiments, the indicated salt was added to the DNA-Cy3(tm) solution that was then applied to the substrate and dried. 20 μl of solution was applied to a piece of 10×12 mm silkscreen. After drying, the actual concentration of salt on the sensor is unknown, but is estimated to be much higher.

The following anions are also useful in testing the nucleic acids for their responses to analytes in vapor phase: Cl (already used in our tests); NO₃ (already used); SO₄ (already used).

The references cited herein and throughout the specification are herein incorporated by reference in their entirety. The examples above, are meant to provide guidance in making and using the present invention, however, the invention is meant to cover all the equivalents of these preferred embodiments which one skilled in the art is capable of preparing based upon this disclosure.

### REFERENCES

Aathithan, S., Plant, J. C., Chaudry, A. N., and French, G. L. (2001). Diagnosis of bacteriuria by detection of volatile organic components in urine using an automated headspace analyzer with multiple conducting polymer sensors. J. Clin. Microbiol., 39:2590-2593.
Alkasab, T. K, Bozza, T. C., Cleland, T. A., Dorries, K. M., Pearce, T. C., White, J., and Kauer, J. S. (1999). Characterizing complex chemosensors: Information theoretic analysis of olfactory systems. Trends Neurosci., 22:102-108.
Alkasab, T. K., White, J., and Kauer, J. S. (2002). A computational system for simulating and analyzing arrays of biological and artificial chemical sensors. Chem. Senses, 27:261-275.
Bartlett, P. N. and Gardner, J. W. (1992). Odour sensors for an electronic nose. In Gardner, J. W. and Barlett, P. N., editors, Sensors and Sensory Systems for an Electronic Nose, pages 31-51. Kluwer Academic Publishers, Dordrecht, Netherlands.
Bock, L. C., Griffin, L. C., Latham, J. A., Vermaas, E. H., and Toole, J. J. (1992). Selection of single-stranded DNA molecules that bind and inhibit human thrombin. Nature, 355:564-566.
Bouche, M. P., Lambert, W. E., Bocxlaer, J. F. V., Piette, M. H., and Leenheer, A. P. D. (2001). Quantitative determination of n-propane, iso-butane, and n-butane by headspace GC-MS in intoxications byinhalation of lighter fluid. J. Anal. Toxicol., 26:35-42.
Buck, L. and Axel, R. (1991). A novel multigene family may encode odorant receptors: A molecular basis forodor recognition. Cell, 65:175-187.
Cam, D. and Gagni, S. (2001). Determination of petroleum hydrocarbons in contaminated soils using solid-phase microextraction with gas chromatography-mass spectroscopy. J. Chromatogr. Sci., 39:481-486.
Cancho, B., Ventura, F., and Galceran, M. T. (2002). Determination of aldehydes in drinking water usingpentafluorobenzylhydroxylamine derivatization and solid-phase microextraction. J. Chromatogr., 943:1-13.
Chen, S., Mahadevan, V., and Zieve, L. (1970). Volatile fatty acids in the breath of patients with cirrhosis oftheliver. J. Lab. Clin. Med., 75:622-627.
Christensen, T. A. and White, J. (2000). Representation of olfactory information in the brain. In Finger, T. E., Silver, W. L., and Restrepo, D., editors, Neurobiology of Taste and Smell, pages 201-232. John Wiley & Sons, New York.
Dickinson, T. A., White, J., Kauer, J. S., and Walt, D. R. (1998). Current trends in ‘artificial-nose’ technology. Trends Biotechnol., 16:250-258.
Ellington, A. D. and Szostak, J. W. (1990). In vitro selection of RNA molecules that bind specific ligands. Nature, 346:818-822.
Freund, M. S. and Lewis, N. S. (1995). A chemically diverse conducting polymer-based “electronic nose”. Proc. Nat. Acad. Sci. USA, 92:2652-2656.
Gardner, J. W. and Barlett, P. N. (1994). A brief history of electronic noses. Sensors and Actuators B, 18-19:211-220.
Gardner, J. W. and Bartlett, P. N., editors (1992). Sensors and sensory systems for an electronic nose, volume 0. Kluwer Academic Publishers, Dordrecht, The Netherlands.
Gardner, J. W. and Hines, E. L. (1997). Pattern analysis techniques. In Kress-Rogers, E., editor, Handbook of Biosensors and Electronic Noses: Medicine, Food, and the Environment, pages 633-652. CRC Press, BocaRaton, Fla.
Gelmont, D., Stein, R. A., and Mead, J. F. (1981). Isoprene—the main hydrocarbon in human breath. Biochem. Biophys. Res. Commun., 99:1456-1460.
George, V., Jenkins, T., Leggett, D., Cragin, J., Phelan, J., Oxley, J., and Pennington, J. (1999). Progress ondete mining the vapor signature of a buried landmine. In Proceedings of the 13th Annual International Symposium on Aerospace/Defense Sensing, Simulation, & Controls, pages 258-269.
George, V., Jenkins, T., Phelan, J., Leggett, D., Oxley, J., Webb, S., Miyares, P., Cragin, J., Smith, J., andBerry, T. (2000). Progress on determining the vapor signature of a buried landmine. In Proceedings of the 14th Annual Interndtional Synposium on Aerospace/Defense Sensing, Simulation, & Controls.
Grate, J. W., Rose-Pehrsson, S. L., Venezky, D. L., Klusty, M., and Wohltjen, H. (1993). Smart sensor system for trace organophosphorus and organosulfur vapor detection employing a temperature-controlled arrayof surface acoustic wave sensors, automated sample preconcentration, and pattern recognition. Anal. Chem., 65:1868-1881.
Grote, C. and Pawliszyn, J. (1997). Solid-phase microextraction for the analysis of human breath. Anal. Chem., 69:587-596.
Hamaguchi, N., Ellington, A., and Stanton, M. (2001); Aptamer beacons for the direct detction of proteins. Anal. Biochem., 294:126-131.
Hartell, M., Myers, L., Waggoner, L., Hallowell, S., and Petrousky, J. (1998). Design and testing of a quantitative vapor delivery system. In Proceedings of the 5th International Symposium on the Analysis and Detection of Explosives, Washington, D.C.: Treasurey Department.
Jayasena, S. D. (1999). Aptamers: An emerging class of molecules that rival antibodies in diagnostics. Clin. Chen., 45:1628-1650.
Jenkins, T. F., Leggett, D. C., Miyares, P. H., Walsh, M. E., Ranney, T. A., Cragin, J. H., and George, V.(2001). Chemical signatures of TNT-filled land mines. Talanta, 54:501-513.
Jenkins, T. F., Walsh, M. E., Miyares, P. H., Kopczynski, J. A., Ranney, T. A., George, V., Pennington, J. C., and T. E. Berry, J. (2000). Analysis of explosives-related chemical signatures in soil samples collected near buried land mines. ERDC TR-00-5, CRREL.
Jhaveri, S., Rajendran, M., and Ellington, A. D. (2000a). In vitro selection of signaling aptamers. Nature Biotech., 18:1293-1297.
Jhaveri, S. D., Kirby, R., Conrad, R., Maglott, E. J., Bowser, M., Kennedy, R. T., Glick, G., and Ellington, A. D. (2000b). Designed signaling aptamers that transduce molecular recognition to changes influorescence intensity. J. Am. Chem. Soc., 122:2469-2473.
Kauer, J. S. (1987). Coding in the olfactory system. In Finger, T. E. and Silver, W. L., editors, Neurobiology of Taste and Smell, pages 205-231. John Wiley & Sons, Inc, New York.
Kauer, J. S. (1991). Contributions of topography and parallel processing to odor coding in the vertebrate olfactory pathway. Trends Neurosci, 14:79-85.
Kauer, J. S. and White, J. (2002). Representation of odor information in the olfactory system: from biology to an artificial nose. In Barth, F. G., Humphrey, J. A. C., and Secomb, T. W., editors, Sensors and Sensing in Biology and Engineering. Springer-Verlag, Berlin.
Kent, P. F. and Mozell, M. M. (1992). The recording of odorant-induced mucosal activity patterns with avoltage-sensitive dye. J Neurophysiol, 68:1804-1819.
Kundu, S. K., Bruzek, J. A., Nair, R., and Judilla, A. M. (1993). Breath acetone analyzer: Diagnostic toolto monitor dietary fat loss. Clin. Chem., 39:87-92.
Lambropoulou, D. A. and Albanis, T. A. (2001). Optimization of headspace solid-phase microextraction conditions for the determination of organophosphorus insecticides in natural waters. J. Chromatogr., 922:243-255.
Leggett, D. C., Cragin, J. H., Jenkins, T. F., and Ranney, T. (2001). Release of explosive-related vapors fromland mines. ERDC TR-01 6, CRREL.
Lu, X.-C. M., Slotnick, B. M., and Silberberg, A. M. (1993). Odor matching and odor memory in the rat. Physiol. Behav., 53:795-804.
MacKay-Sim, A., Shaman, P., and Moulton, D. G. (1982). Topographic coding of olfactory quality: Odorant-specific patterns of epithelial responsivity in the salamander. J. Neurophysiol., 48:584-596.
Mendis, S., Sobotka, P. A., and Euler, D. E. (1994). Pentane and isoprene in expired air from humans: Gas-chromatographic analysis of single breath. Clin. Chem., 40:1485-1488.
Mitchell, S., Ayesh, R., Barrett, T., and Smith, R. (1999). Trimethylamine and Foetor Hepaticus. Scand. J. Gastroenterol, 34:524-528.
Moore, D. (2001). Preparation and analysis of dna. In Ausubel, F. M., Brent, R., Kingston, R. E., Moore, D. D., Seidman, J. G., Smith, J. A., and Struhl, K., editors, Current Protocols in Molecular Biology, volume 1, chapter 2. John Wiley & Sons, Inc.
Mosaddegh, M. H., Richardson, R:, Stoddart, R. W., and McClure, J. (2001). Application of solid-phase micro-extraction technology to drug screening and identification. Ann. Clin. Biochem., 38:541-547.
Musshoff, F., Junker, H., and Madea, B. (2002). Simple determination of 22 organophosphorous pesticides in human blood using headspace solid-phase microextraction and gas chromatography with massspectrometric detection. J. Chromatogr. Sci., 40:29-34.
Narasimhan, L. R., Goodman, W., and Patel, C. K. N. (2001). Correlation of breath ammonia with blood ureanitrogen and creatinine during hemodialysis. Proc. Natl. Acad. Sci., 98:4617-4621.
Park, J., Groves, W. A., and Zellers, E. T. (1999). Vapor recognition with small arrays of polymer-coated microsensors. a comprehensive analysis. Anal. Chem., 71:3877-3886.
Passe, D. H. and Walker, J. C. (1985). Odor psychophysics in vertebrates. Neurosci. Biobehav. Rev., 9:431-467.
Persaud, K. and Dodd, G. (1982). Analysis of discrimination mechanisms in the mammalian olfactory system using a model nose. Nature, 299:352-355.
Phillips, M., Gleeson, K., Hughes, J. M. B., Greenberg, J., Cataneo, R. N., Baker, L., and McVay, W. P. (1999). Volatile organic compounds in breath as markers of lung cancer: a cross-sectional study. Lancel, 353:1930-1933.
Ping, W., Yi, T., Haibao, X., and Farong, S. (1997). A novel method for diabetes diagnosis based on electronic nose. Biosens. Bioelectron., 12:1031-1036.
Potyrailo, R. A., Conrad, R. C., Ellington, A. D., and Hieftje, G. M. (1998). Adapting selected nucleic acidligands (aptamers) to biosensors. Anal. Chem., 70:3419-3425.
Sassanfar, M. and Szostak, J. W. (1993). An RNA motif that binds ATP. Nature, 364:550-553.
Schneider, J. F., Boparai, A. S., and Reed, L. L. (2001). Screening for Sarin in air and water by solid-phase microextraction-gas chromatography-mass spectrometry. J. Chromatogr. Sci., 39:420-424.
Severin, E. J., Doleman, B. J., and Lewis, N. S. (2000). An investigation of the concentration dependence andresponse to analyte mixtures of carbon black/insulating organic polymer composite vapor detectors. Anal. Chem., 72:658-668.
Sidebothiam, R. L. and Baron, J. H. (1990). Hypothesis: Helicobacter pylori, urease, mucus, and gastric ulcer. Lancet, 335:193-195.
Slotnick, B. M., Kufera, A., and Silberberg, A. M. (1991). Olfactory learning and odor memory in the rat. Physiol. Behav., 50:555-561.
Tangerman, A., Meuwese-Arends, M. T., and Jansen, J. B. M. J. (1994). Cause and composition of foetor hepaticus. Lancet, 343:483.
Tassopoulos, C. N., Barnett, D., and Fraser, T. R. (1969). Breath-acetone and bloqd-sugar measurements indiabetes. Lancet, 1(7609):1282-1286.
Tuerk, C. and Gold, L. (1990). Systematic. evolution of ligands by exponential enrichment: RNA ligands tobacteriophage T4 DNA polymerase. Science, 249:505-510.
Tyagi, S. and Kramer, F. R. (1996). Molecular beacons: Probes that fluoresce on hybridization. Nature Biotech., 14:303-308.
White, J., Dickinson, T. A., Walt, D. R., and Kauer, J. S. (1998). An olfactory neuronal network for vaporrecognition in an artificial nose. Biol. Cybern., 78:245-251.
White, J. and Kauer, J. S. (1999). Odor recognition in an artificial nose by spatio-temporal processing usingan olfactory neuronal network. Neurocomputing, 26-27:919-924.
White, J., Kauer, J. S., Dickinson, T. A., and Walt, D. R. (1996). Rapid analyte recognition in a device basedon optical sensors and the olfactory system. Anal. Chem., 68:2191-2202.
Yamazaki, K., Singer, A., and Beauchamp, G. K. (1998-1999). Origin, functions and chemistry of h-2 regulated odorants. Genetica, 104:235-240.
Yang, J. S. and Swager, T. M. (1998). Porous shape persistent fluorescent polymer films: An approach toTNT sensory materials. J. Am. Chem. Soc., 120:5321-5322.
Youngentob, S. L., Markert, L. M., Mozell, M. M., and Hornung, D. E. (1990). A method for establishing afiveodor identification confusion matrix task in rats. Physiol. Behav., 47:1053-1059.
Zellers, E. T., Batterman, S. A., Han, M., and Patrash, S. J. (1995). Optimal coating selection for the analysis of organic vapor mixtures with polymer-coated surface acoustic wave sensor arrays. Anal. Chem., 67:1092-1106.
Zhang, Z., Yang, M. J., and Pawliszyn, J. (1994). Solid-phase microextraction. Anal. Chem., 66:844A-853A.

1. A method for detecting an analyte in an air sample comprising the steps of:
a. contacting said air sample with a nucleic acid/fluorophore-based sensor array comprising a substrate; and a nucleic acid labeled with a fluorophore dispersed on said substrate, said nucleic acid labeled with a fluorophore providing a characteristic optical response when subjected to excitation light energy in the presence of the analyte; and
b. detecting the presence or absence of said analyte.

2. The method of claim 1, wherein said nucleic acid is dispersed on a plurality of internal and external surfaces said substrate.

3. The method of claim 1, wherein said contacting further comprises drawing an air sample believed to contain said analyte into a sample chamber and exposing said nucleic acid/fluorophore based sensor array to said air sample.

4. The method of claim 1, wherein said detecting further comprises:
a. illuminating said nucleic acid/fluorophore based sensor array with excitation light energy; and
b. measuring an optical response produced by said nucleic acid/fluorophore based sensor array due to the presence of said analyte with a detector means.

5. The method of claim 4, further comprising identifying said analyte by employing a pattern-matching algorithm; and comparing said optical response of said nucleic acid/fluorophore based sensor array with said characteristic optical response.

6. The method of claim 4, further comprising identifying said analyte by providing spatio-temporal response patterns of said optical response; and recognizing said patterns through a method selected from the group consisting of a template matching, neural networks, delay line neural networks, and statistical analysis.

7. The method of claim 1, wherein the air sample is suspected of containing explosive materials.

8. The method of claim 1, wherein the air sample is suspected of containing a chemical weapons agent.

9. A method of selecting a nucleic acids capable of responding to a vapor phase analyte, said method comprising:
a. contacting the nucleic acid labeled with a fluorophore with an analyte in vapor phase; and
b. measuring the emission proflile of the fluorophore in the presence and absence of the target analyte, wherein a difference in the emission profile indicates that the nucleic acid is responsive to the analyte in vapor phase.

10. The method of claims 1, 4 and 9, wherein the nucleic acid is 1-3000 bases long.

11. The method of claims 1, 4 and 9, wherein the nucleic acid is 10-500 bases long.

12. The method of claims 1, 4 and 9, wherein the nucleic acid is 15-24 bases long.

13. The method of claims 1, 4 and 9, wherein the fluorophore is attached to the 3′ region or a 5′ region of the nucleic acid.

14. The method of claims 1, 4 and 9, where the nucleic acid is internally labeled with the fluorophore.

15. The method of claim 14, wherein the fluorophore is an applied dye.

16. The method of claim 15, wherein the applied dye is YO-PRO or OliGreen.

17. The method of claims 1, 4 and 9, wherein the substrate is a silk screen.

18. The method of claims 1 4 and 9, wherein the substrate is glass.

19. A sensing system for detecting and identifying a volatile compound in an air sample comprising:
a. a nucleic acid/fluorophore based sensor array comprising a plurality of nucleic acids;
b. a fluorophore attached to said nucleic acids;
c. a plurality of substrates wherein said nucleic acids are attached to;
d. a substrate support; e. an excitation light source array comprising a plurality of light sources optically coupled to said sensor elements;
f. a detector array comprising a plurality of detectors optically coupled to said sensor elements; g. a sample chamber for housing said sensor elements, said light source array, said detector array;
h. a sampling means enclosed in said chamber for drawing said ambient air into said chamber for contact with said nucleic acid/fluorophore based sensor array for a controlled exposure time;
i. a controller means in electrical communication with said light sources, said detectors, and said sampling means, said controller means electrically coordinating and switching said sampling means with said light sources and said detectors for sampling said ambient air, measuring optical responses of said nucleic acid/fluorophore based sensor arrays to said ambient air sample, and detecting said volatile compound; and
j. an analyte identification algorithm for comparing said measured sensor optical responses to characteristic optical responses of said sensors to target analytes and identifying said analyte in said air sample.

20. A sensing system for intelligent detecting and identifying an analyte in an air sample comprising:
a. a nucleic acid/fluorophore based sensor array comprising a plurality of nucleic acids attached to a fluorophore;
b. a detector array comprising a plurality of detectors in communication with said nucleic acid/fluorophore based sensor array;
c. a sampling chamber for housing said nucleic acid/fluorophore based sensor array and said detector array;
d. a sampling means enclosed in said chamber for drawing said ambient air into said chamber for contact with said nucleicacid/fluorophore based sensor array for a controlled exposure time;
e. a microcontroller in communication with said sampling means and said detector array, said controller means coordinating and switching said sampling means and said detector array for sampling said ambient air, measuring responses of said sensors to said air sample, detecting said analyte, and reporting an analyte detection result;
f. a sampling algorithm for directing said microcontroller; and
g. an analyte identification algorithm in communication with said sampling algorithm and said microcontroller, said identification algorithm comparing said measured sensor optical responses before and after exposure to the analyte to characteristic responses of said sensors to analytes and identifying said analyte in said air sample.

21. The sensing system of claim 20, wherein said identification algorithm comprises a response report comparing a spatio-temporal pattern of said measured optical responses to a spatio-temporal pattern of said characteristic responses; and an identification report selected from the group consisting of a pattern match, a delay line neural network match, and a neuronal network match.

22. The sensing system of claim 20, wherein the sensing system is attached to a shipping container.

23. The sensing system of claim 20, wherein the sensing system is attached to an x-ray screening machine.

24. The sensing system of claim 20, wherein the sensing system is remotely controllable.

25. The sensing system of claim 20, wherein the sensing system is incorporated into. a hand-held device.

26. A sensor array system for remote characterization of a gaseous or vapor, sample, comprising:
a. a plurality of sensors, wherein at least one sensor comprises nucleic acid/fluorophore combination comprising a plurality of nucleic acids attached to a fluorophore, wherein the plurality of sensors provide -a detectable signal when contacted by an analyte;
b. a measuring apparatus, in communication with plurality of sensors capable of measuring the detectable signal;
c. a transmitting device, in communication with the measuring apparatus for transmitting information corresponding to the detectable signal to a remote location via the Internet, fiber optic cable, and/or an air-wave frequency; and a computer comprising a resident algorithm capable of characterizing the analyte.

27. The sensor array system according to claim 23, wherein the sensor system comprises a plurality of measuring apparatuses.
